(19) United States
(12) Patent Application Publication
(10) Pub. No.: US 2025/0361520 A1
(43) Pub. Date: Nov. 27, 2025

(54) METHODS AND COMPOSITIONS FOR PRODUCING SELF-AMPLIFYING RNA FOR GENE SILENCING IN PLANTS

(71) Applicant: UNIVERSITY OF FLORIDA RESEARCH FOUNDATION, INCORPORATED, Gainesville, FL (US)

(72) Inventor: Dean W. GABRIEL, Gainesville, FL (US)

(21) Appl. No.: 18/873,854

(22) PCT Filed: Jun. 14, 2023
(86) PCT No.: PCT/US2023/025251

§ 371 Date: Dec. 11, 2024

Related U.S. Application Data
(60) Provisional application No. 63/351,931, filed on Jun. 14, 2022.

(51) Int. Cl. C12N 15/82; C12N 9/12; C12N 15/113; C12N 15/88

(52) U.S. Cl. CPC C12N 15/8281; C12N 9/127; C12N 15/113; C12N 15/8282; C12N 15/88; C12Y 207/07048; C12N 2310/14

(57) ABSTRACT

The present invention teaches methods and compositions useful for treating, preventing, or curing pathogen infections of living plants. In particular, the present invention teaches methods of enhancing plant response to pathogen-associated molecular patterns using self-amplifying RNA expressing a dsRNA. The methods and compositions described herein are effective at treating biotrophic pathogens, including Liberibacters.

### GOVERNMENT SUPPORT

This invention was made with government support under Cooperative Agreement 5 AP18PPQS&T00C131 awarded by the United States Department of Agriculture. The government has certain rights in the invention.

### FIELD

The present disclosure relates to methods and compositions for preventing, eliminating, reducing, or otherwise ameliorating infections and/or damage of crop plants by use of RNA interference against fungal or bacterial plant pathogens.

### DESCRIPTION OF THE TEXT FILE SUBMITTED ELECTRONICALLY

The contents of a text file submitted electronically herewith are incorporated herein by reference in their entirety: A computer readable format copy of the Sequence Listing (filename: 10457531PC0_sequncelisting.xml, created Jun. 13, 2023, with a size of 33000 bytes).

### BACKGROUND

All animal and plant cells have highly regulated cell defense responses and suicide programs designed to limit the damage done to one cell or a group of cells from affecting the entire organism. This is why cells die after radiation damage from sunburn, for example; otherwise, the radiation damage would result in mutations that might result in cancers, or in skin tissue with greatly aged appearance and performance. This suicide program is tightly controlled in all organisms, and it requires a combination of factors to come together to trigger the cell death program. Once initiated, it is irreversible. Permanent suppression of these regulated brakes on cell defense/death by mutations is typically lethal. Transient suppression of these brakes, on the other hand, can increase defenses. The level of suppression is critical to practical disease control success.

Most pathogens have evolved mechanisms to avoid triggering plant cell death programs, and defense responses. A solution for regaining control of cell death/defense mechanisms by transiently suppressing specific host genes is provided by way of RNA interference (RNAi). This can be done in plants by spray application of double stranded RNA (dsRNA) solutions; however, the cost to produce dsRNA is prohibitive on an agricultural scale, and the short duration of effects requires multiple applications per growing season. Increasing the efficacy and duration of effects of the applied RNA is needed to address the emergence and proliferation of and/or damage caused by these pathogens.

### SUMMARY

The present disclosure teaches compositions and methods useful for protecting plants against both intracellular and intercellular bacterial and fungal attack, growth and infection, comprising the silencing of selected plant target genes by transient RNA interference (RNAi). More specifically, disclosed is a platform technology enabling an application of self-amplifying RNA (SAM) that produces much higher levels of RNAi in plants than applied dsRNA alone. The self-amplifying RNA is designed to repress activity of one or more plant or insect vector genes. Double stranded RNA (dsRNA) is recognized by the eukaryotic enzyme Dicer (RNAse III) and cleaved, creating small interfering RNAs (siRNAs) of about 21-mer length. In plants, these siRNAs are phloem mobile. Each 21-mer fragment is unwound by helicases to form single stranded 21-mers, but only the antisense strand is incorporated into the RNA-induced silencing complex (RISC). The other is discarded. The siRNA/RISC complex can then specifically cleave or block expression of messenger RNA (mRNA), thereby silencing the target gene. The primary disadvantage of dsRNA sprays for large scale field applications is the cost of RNA synthesis. A second disadvantage for this purpose is lack of persistence of the effect. That is, the effects are too transient.

In plants and Caenorhabditis elegans, siRNAs may be amplified by a host RNA-dependent RNA polymerase (RdRp). Many eukaryotic viruses also encode RdRps that are self-amplifying (SAM), along with certain replaceable subgenomic regions operationally linked to the viral RdRp, including grapevine virus A (GVA), NCBI Reference Sequence: NC_003604.2. We had the genes encoding GVA RdRp and a subgenomic region encoding Green Fluorescent Protein (GFP) synthesized commercially (refer to Example 1 and schematic in FIG. 1). For proof of concept that the GVA RdRp could express a gene in a plant species other than Vitis vinifera grapevines, the SAM genes were expressed in vitro, capped, and the RNA encoding GFP was transformed into tobacco protoplasts. The GFP protein was highly expressed from the subgenomic region operationally linked to the GVA RdRp in nonhost tobacco protoplasts (FIG. 2), thereby providing proof of concept. From 1-2% of the protoplasts glowed very brightly from the added RNA, indicating both self-amplification and good gene expression in tobacco (FIG. 2).

dsRNAs were redesigned for siRNA purposes to insert into the subgenomic region downstream of the RdRp to form transcriptional fusions and replacing GFP. These fusions created a SAM that formed dsRNA from the subgenome, that in turn would be diced and phloem mobile (FIG. 3). The dsRNA regions of these SAMs were chimeric genes developed from sequences taught in earlier patent applications (ie., PCT/US2015/062698 and PCT/US2019/048870) to have some disease control effect in commercial citrus field trials due to siRNA silencing of citrus host genes. The original dsRNAs were randomly selected large (300-500 bp) blocks of the target gene mRNA coding regions, synthesized in large quantities commercially and directly applied to citrus. In order to improve upon the use of randomly selected large random blocks of dsRNA for siRNA production, we also designed large (300-500 bp) chimeras of small (21 bp) blocks selected from the target mRNA coding region using siRNA-Finder (Si-Fi) software (Luck et al., 2019). These dsRNA chimeras were synthesized commercially in large quantities and directly applied to citrus in plain water (refer Example 2), and as aqueous solutions encapsulated in negatively charged (Zeta potential-45.67 at pH 7) nanoemulsions consisting of nanoparticles formed of lecithin, gelatin and dsRNA. The particles ranged in size from 72 to 296 nm, with a major peak of 144 nm (FIG. 4).

The purpose of forming the lecithin coated nanoemulsions was to try to improve plant cellular uptake of the dsRNA since such emulsions are known to become positively charged at acidic pH (Pérez et al., 2012), and citrus phloem has an acidic pH of 6.0 (Hijaz & Killiny 2014). The cellular uptake of these dsRNA chimeras was greatly enhanced by this cationic lecithin coating in acidic citrus phloem, since RNA itself is highly anionic as well as hydrophilic, which is known to inhibit cellular uptake in animal cells (Pérez et al., 2012). Finally, we synthesized a gene encoding both sense and antisense strands of an improved chimeric RNA sequence to replace GFP in the subgenomic region driven by GVA RdRP. The sense and antisense strands were designed to cause the transcript to form a hairpin loop, thus creating a SAM that formed dsRNA from the subgenome of the expressed RNA that could be diced (FIG. 3). In vitro transcription reactions were performed to create mRNA, and the mRNAs were capped. Aqueous solutions of the SAM encoding the previously field trial tested dsRNA was injected into citrus, either formed into lecithin nanoemulsions as described, or directly after capping and without further treatment. In both cases, the SAM appeared to self-replicate in citrus, and the expressed dsRNA appeared to be diced, since the small 21 mers became demonstrably phloem mobile and were readily detected in non-inoculated leaves in distant parts of inoculated citrus trees within 2 days, maximized at 7 days, and persisted for at least 14 days.

The present disclosure relates to methods and compositions for preventing, reducing, eliminating or otherwise ameliorating infections and/or damage of crop plants by bacterial and fungal pathogens. The percentage reduction in pathogen infection and/or plant damage for plants protected using the compositions and methods of the disclosed embodiments is at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% greater/better when compared to an appropriate control or check plant grown under the same plant husbandry conditions. The amount of pathogen infection and/or plant damage can be measured using methods well known to those skilled in the art. Plant infection, e.g., can be measured as the percentage of necrotic tissue on the plants. Plant damage, e.g., can be measured as total yield of a specific plant part (e.g., number or weight of seeds, number or weight of pods, plant weight, plant height, number of weight of flowers, root mass measured in volume or by weight, etc.). An appropriate control or check plant is one in which the targeted gene(s) have not been silenced as they are silenced in the test plant(s) according to the compositions and methods of the present invention.

Although SAM vaccines based on an alphavirus genome have become a platform technology for eliciting animal cell immune responses by way of protein antigen expression (for example, see Lou et al., 2020 and references therein), there is no teaching or suggestion that a plant alphavirus could be used to create self amplifying dsRNA in plants for the purpose of silencing one or more plant genes at distant locations in commercial trees. Specifically, the present disclosure teaches use of SAM to do just that, and to enhance the longevity of effect of applied dsRNA, including chimeric dsRNAs, from one to two weeks and reduce the level of applied RNA needed by at least 1,000 fold, and still retain the original RNAi effect.

In one embodiment, the specific plant target is a tomato gene. In another embodiment, the specific plant target is a citrus gene, specifically of Citrus sinensis cultivar Valencia. In another embodiment, the target gene is a grapefruit gene (C. paradisi). In other embodiments, the specific target is from potato, tobacco, celery, pear, apple, plum, cherry, olive, or Vitis vinifera grapes.

Given the current and growing availability of genomic DNAs, multiple plant and insect vector genes can now readily be identified and targeted for silencing by those skilled in the art from virtually any plant or insect vector source for which a DNA sequence is available using a PCR cloning strategy similar to that used here, including woody species such as Malus domestica apple, Theobroma cacao cocoa, Prunus persica peach, Populus deltoides poplar, Olea europaea olive, vines such as Vitis vinifera grape, and agronomic crop plants such as Gossypium hirsutum cotton, Glycine max soybean, Arabidopsis, Solanum tuberosum potato, Solanum lycopersicum tomato, Nicotiana tobacco and many others. Further, insect vectors that feed on plants, such as the Asian citrus psyllid, Diaphorina citri, have also been sequenced and their genes can readily be targeted for silencing using the methods taught here. In further embodiments, any segment, section or part of the full length genomic mRNAs from any of these species can be used to silence genes of nearly identical sequence in a wide variety of related strains and cultivars of that species, including both the 5′ and 3′ untranslated regions (i.e., not only the fragments currently deposited in GenBank).

The present disclosure also provides compositions and methods for the protection and/or curing of plants from infections caused by biotrophic bacteria and fungi by complete or partial (i.e., incomplete) suppression of targeted homologs. In one embodiment, the invention provides compositions and methods for the protection of citrus cells from infection by biotrophs. In some embodiments, the invention provides compositions and methods for the protection and curing of phloem cells of Citrus, Solanaceous, and other plant families infected by various species of the bacterial pathogen genus Liberibacter. In some embodiments, the invention provides compositions and methods for the protection and curing of citrus phloem from infection by Ca. Liberibacter asiaticus (Las).

The present disclosure builds upon compositions and methods for the protection and curing of commercial crops, including tree crops (e.g. citrus trees) that are diseased by an infectious agent by using a polynucleotide spray, including, e.g., via a SAM that creates RNA that can be used to created phloem mobile, siRNAs that can transiently suppress expression of one or more plant or insect target genes. When dsRNA spray is applied to plants, the silencing effect on the target gene occurs within 2-3 days, and the effect lasts for up to 3 weeks (de Andrade & Hunter, 2016). That is, such silencing is transient, and not permanent. However, dsRNA is expensive to produce and apply on an agricultural scale. In certain embodiments, disclosed is the use of a SAM that forms a dsRNA from an expressed subgenomic portion of the SAM, the effects of which occur faster (within 1 day), last longer (for up to 4 weeks), and critically, 1,000× less RNA is required. Such SAM mediated silencing is also transient. In addition, in some embodiments, the SAM is encapsulated to increase its entry into living phloem cells. In some embodiments, provided are compositions and methods for the protection and curing of citrus phloem cells from infection by Liberibacters, including CLas,

In some embodiments, the present disclosure teaches that a SAM can form dsRNA about 20, 21, 22, 23, 24, 25, 26, 27, 28, 29, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 base pairs in length. The SAM transcript can induce RNAi and suppress target gene expression in plants citrus when applied as sprays from the outside of the citrus plant. In some embodiments well known to those skilled in the art, the RNAi can be induced not only by application of dsRNA, but by any double stranded polynucleotide, including synthetic polynucleotides. In some embodiments well known to those skilled in the art, antisense polynucleotides can be formed not simply from polynucleotides, but also from phosphorodiamidate morpholino oligomers (PMOs).

In some embodiments, the disclosed are compositions and methods for delivering SAM encoding a dsRNA into phloem cells using nanoemulsions that stabilize the SAM and facilitate its uptake, which is then effective in repressing, preventing or otherwise reducing bacterial or fungal infections of a plant comprising expressing an antisense or RNA interference construct based on a plant protein or nucleic acid sequence.

In some embodiments, the present invention teaches that similar RdRPs may be used from other sources, plant or viral. In some embodiments, the present disclosure teaches the use of RdRP genes with at least 70%, or at least 80%, or at least 90%, or at least 95%, or at least 98% sequence identity to the RdRp sequence disclosed in SEQ ID NO: 1. These RdRp genes are used to drive subgenomic sequences that are either antisense alone or both sense and antisense, engineered with a loop to form a dsRNA hairpin structure that will be diced to form siRNAs as illustrated on the left side of FIG. 5. Such silencing sequences can be comprised of long random stretches from untranslated and/or translated regions of an mRNA, or a chimeric tandem array of 21-22-mers from untranslated and/or translated regions of an mRNA, as illustrated on the left side of FIG. 5. In some embodiments, disclosed herein is the use of siRNAs derived from different gene targets that are synthesized to form tandem chimeras of various lengths that are stacked against different target genes. In some embodiments, the present invention teaches the use of siRNAs derived from different portions of the same gene target that are synthesized to form tandem chimeras of various lengths, in one case the target gene is SSADH (GenBank XM_006493686) and an example of a chimera synthesized as dsRNA and based on SSADH is exemplified in SEQ ID NO: 2. In some embodiments, the present invention teaches the formation of subgenomic dsRNA of a SAM in size from about 200 to about 2,000 bp in length. In one embodiment, the target gene is SSADH (GenBank XM_006493686) and an example of a chimera synthesized as a subgenomic DNA that is expressed to form a hairpin loop for RNAi is exemplified in SEQ ID NO: 3. In some embodiments, the RNAi is achieved by topical spray applications of the SAM and dsRNA subgenome. In some embodiments, the RNAi is achieved by the SAM and dsRNA subgenome applied by spray, laser etching or mechanical penetration of leaf and stem cuticle layers using the SAM. In some embodiments, the RNAi is achieved by encapsulating the SAM and dsRNA subgenome into a nanoemulsion. In some embodiments, the RNAi is achieved by coating the applied by the SAM and dsRNA subgenome formed into a nanoemulsion comprised of lecithin.

In some embodiments, the methods of the present invention increase plant resistance to at least one biotrophic pathogen. In certain embodiments, the biotrophic pathogens of the present invention are Liberibacters.

### BRIEF DESCRIPTION OF THE DRAWINGS

FIG. 1. Concept of alpha virus self-amplification along with sub-genome region, after cell transformation. RNA-dependent RNA polymerase (RdRp) genes and subgenome expression. GOI=gene of interest. Diagram after Geall et al., 2012.

FIG. 2. Proof-of-Concept that grapevine virus A (GVA) genome could be used to express green fluorescent protein (GFP) in tobacco plant cells. The GVA genes were commercially synthesized, its mRNA expressed in vitro, capped, and the capped mRNA used to transform N. benthamiana protoplasts. Fifteen minutes after addition of the mRNA, matching photos were taken. Left side is 400× view through fluorescence microscope; right side is simple Brightfield view of identical slide, again 400×, with fluorescence off. Far right is higher level magnification of protoplasts in process of breaking. Yield of fluorescent protoplasts, ca 2%.

FIG. 3. Grapefruit target gene (SSADH) suppression using 0.84 g per tree of dsRNA applied by spray. Analysis of variance (ANOVA and Tukey-Kramer tests both indicated significant effect on the target gene.

FIG. 4. Size range and Zeta potential of nanoemulsions formed from lecithin, gelatin and dsRNA at pH 7.

FIG. 5. Phloem siRNA design for GV-RNA-IPGIMv4. Chimeras that were predicted to result in siRNAs were designed based on various exon regions of one or more citrus target genes and the sequences were assembled in tandem array and synthesized, forming the “IPG-1” chimera that may be directly synthesized as dsRNA (SEQ ID NO: 2) This chimera was modified, duplicated and inverted with a loop added to form a dsRNA (SEQ ID NO: 3) from the subgenomic region of the RdRp of modified grapevine virus A, GV-RNA-IPG1Mv4 (SEQ ID NO: 4). The mRNA of this construct was expressed in vitro, capped, and the capped mRNA injected into the spongy mesophyll of leaves of ca. 2 month old, 1.5 foot tall young sweet orange citrus plants in pots.

FIG. 6. Experimental set up for detection of movement of specific siRNA in citrus sweet orange plants after inoculation using capped mRNA expressed from GV-RNA-IPG1Mv4. Four plants were inoculated by infiltrating the spongy mesophyll area of two leaves in each plant through the stomata. The areas of infiltration were marked with a pen. Five samples were taken from each plant, one within the inoculation zone of an inoculated leaf on each plant, one outside the inoculation zone but on the same inoculated leaf, and 3 more on different uninoculated leaves of each plant; at least one was above and one below the inoculated leave on each plant. Samples were taken on Days 1, 8, 14 and 30.

FIG. 7. Sweet orange target gene (SSADH) suppression using 0.140 g per tree of capped mRNA expressed from IPG1-Mv4, encapsulated in a nanoemulsion formed from lecithin and gelatin and injected into sweet orange using the experimental design shown in FIG. 6. Analysis of variance (ANOVA) and Tukey-Kramer tests both indicated a significant effect on the target gene by two weeks after inoculation.

FIG. 8. The modular DNA structure of IPG1-Mv4. The sense and antisense strands of virtually any target gene can be incorporated in place of Fragments D (FragD) and E (FragE). by swapping.

### DETAILED DESCRIPTION

### Overview

### ROS can Trigger Apoptosis

Type I programmed cell death (PCD) or apoptosis, is a genetically programmed and highly regulated cell death mechanism found in plant and animal cells that allows damaged cells to commit suicide. Apoptosis is critically important for elimination of damaged or infected cells that could compromise the function of the whole organism. Typical triggers of apoptosis are environmental insults or stresses that can damage cells or their DNA content. Reactive oxygen species (ROS), including superoxide anions, hydrogen peroxide, nitric oxide and free hydroxide radicals are produced in response to stress, and particularly stress causing mitochondrial damage (Portt, et al., 2011). ROS production per se is a first line of defense in animal and plant cells against biotic disease agents, such as bacteria and fungi. ROS is also one of the major signals that can trigger apoptosis. In addition to ROS production, stress also activates production of the protein “Bax”, and the sphingolipid “ceremide”, and all three are direct proapoptotic messengers. These three major proapoptic messengers can to act independently of one another, since increases in the levels of any one of them (Bax, ROS, or ceramide) is sufficient to trigger apoptosis, but most often, they appear to act in concert. Pathogens that benefit from plant cell death, such as Phytopthora, Ralstonia, Pseudomonas and Xyella are at least somewhat necrotrophic in lifestyle; that is, they kill host cells in order to provide nutrients to sustain in planta population growth. Such pathogens may do little to suppress apoptosis (type I) or necrotic (type III) programmed cell death (Portt, et al., 2011). Other pathogens, such as the obligate fungal parasites (rusts and mildews) and some bacteria, such as Rhizobium and pathogenic Liberibacters, are biotrophic, and must establish intimate cell membrane to membrane contact using haustoria or infection threads.

### Programmed Cell Death for Controlling Infections

Liberibacters are the ultimate form of biotroph, living entirely within the living host cell and surrounded by host cell cytoplasm. For obligate biotrophs, host cell death is a lethal event. Liberibacters, which must live entirely within living phloem cells, would have no options if their phloem host cells simply died. Biotrophic pathogens typically have multiple mechanisms to suppress apoptosis or necrotic programmed cell death. In the case of necrotrophic pathogens (those that rely on killing plant cells in order to feed on the contents), suppressing host cell death results in denial of nutrients, and resistance is the result. Necrotrophic pathogens naturally trigger PCD.

### Liberibacters and Other Biotrophic Bacteria

Prototrophic pathogens rely on fully functional living cells to survive. For example, all pathogenic species and strains of the genus Liberibacter live in plants entirely within living plant phloem cells. Members of the genus are plant pathogens mostly transmitted by psyllids. The first Liberibacter species described was Candidatus Liberibacter asiaticus (Las), the causal agent of Huanglongbing (HLB), commonly known as citrus “greening” disease. HLB is lethal to citrus and is one of the top three most damaging diseases of citrus. The second species described was found in Africa, Ca. L. africanus (Laf), and the third, Ca. Liberibacter americanus (Lam) was found in Brazil. All three cause HLB in citrus. Beside the three citrus Liberibacters associated with HLB, three non-citrus Liberibacter species have been described. Ca. L. solanacearum (Lso), has been identified as the causal agent of serious diseases of potato (“Zebra chip”), tomato (“psyllid yellows”) and other solanaceous crops in the USA, Mexico, Guatemala, Honduras, and New Zealand (Hansen, et al., 2008; Abad, et al., 2009; Liefting, et al., 2009; Secor, et al., 2009). More recently, a different haplotype of Lso was found infecting carrots in Sweden, Norway, Finland, Spain and the Canary Islands (Alfaro-Fernandez, et al., 2012a, 2012b Munyaneza, et al., 2012a, 2012b; Nelson, et al., 2011). A fifth species of Liberibacter, Ca. L. europaeus (Leu) was recently found in the psyllid Cacopsylla pyri, the vector of pear decline phytoplasma. Finally, a sixth species of Liberibacter, Liberibacter crescens (Lcr), was characterized after isolation from diseased mountain papaya (Babaco). Except for Lcr, which is not known to be pathogenic, all other described Liberibacters are pathogenic and must be injected into living plant cells by specific insects. All are Gram-negative bacteria in the Rhizobiaceae family.

Furthermore, the pathogenic Liberibacters can only live within specific insect and plant cells; as obligate parasites, they do not have a free-living state-they are extreme biotrophs.

As an example of an ordinary biotroph, Xanthomonas citri, which causes citrus canker disease, invades the air spaces within a leaf and relies on inducing cell divisions in living cells in order to rupture the leaf surface (Brunings and Gabriel, 2003). Obviously, for biotrophs, host cell death would be expected to severely limit growth in planta.

### Liberibacter Mechanisms of Avoiding or Suppressing Innate Immunity.

The genomes of Las and Lam differ (among other things) in that most Las strains have 4 copies of peroxidase (Zhang, et al., 2011), and most Lam strains have 2 copies (Wulff, et al., 2014). These are critical lysogenic conversion genes (conferring ability to colonize a plant or insect). With both Las and Lam (and likely Lso), these genes are amplified in copy number on a plasmid prophage to increase transcript copy number, and therefore, protein levels (Zhang, et al., 2011). Peroxidases degrade reactive oxygen species (ROS), like hydrogen peroxide. ROS production is one of the primary insect and plant host defenses against microbes. Since Liberibacters colonize living phloem cells and multiply within the plant cell cytoplasm, the ability to degrade ROS is a critical matter of survival to Liberibacter. In addition to the peroxidase genes, all pathogenic Liberibacters encode two peroxiredoxins on their chromosomes. One of the peroxiredoxins is secreted, and not only degrades ROS, but also travels outside the bacteria to prevent peroxidative degradation of lipid membranes in planta, thereby preventing a chain reaction peroxidation event and subsequent accumulation of antimicrobial oxylipins, that are not only antimicrobial but which can also trigger PCD (Jain et al 2018). Furthermore, this peroxiredoxin also degrades reactive nitrogen species (RNS), attenuates NO-mediated SAR signaling and scavenges peroxynitrite radicals, all of which allow repetitive cycles of infection (Jain et al, 2022).

Both peroxiredoxins and peroxidases actively suppress PCD, and since ROSs and RNSs are strong pro-apoptotic inducers of PCD, particularly under certain nutrient deficiencies, the ability to absorb and degrade ROS and RNS is a matter of survival for bacteria that need to keep their host cells alive. Since Liberibacters can occupy a significant volume of host cell cytoplasm, the ability to absorb and degrade ROS and RNS appears to be critical to suppressing plant and insect vector cell apoptosis.

Plant pathogens provide a series of molecular signals that are detected by the plant and can trigger PCD. These signals, or “pathogen associated molecular patterns” (PAMPs) are detected by plants as alien molecules and trigger strong defense responses called “innate immunity” in plants. Avoidance of triggering PCD by biotrophs involves eliminating by evolution over time, to the greatest possible extent, production of PAMPs.

The genome sequences of all pathogenic Liberibacters are highly reduced in size (all are ca. 1.26 Mb) as compared with their closest Rhizobium relatives (genome sizes>6.4 Mb); significantly, both Las and Lam appear to lack flagella, a known PAMP, although both encode structural genes for flagellin. In addition, Lam lacks most of the genes needed to make lipopolysaccharide (LPS), a particularly potent PAMP and an important defensive barrier molecule integral to the outer membrane of most Gram-negative bacteria.

### Plant Regulators of ROS Production.

Spatiotemporal regulation of ROS generation and detoxification pathways is critical in plants for maintaining redox poise while preventing oxidation of cellular macromolecules (Schieber and Chandel 2014). Failure to maintain redox poise triggers PCD. This invention is based in part on the inventors' discovery that moderate and transient down regulation of expression of the citrus SSADH gene through the use of siRNA resulted in resistance to a biotrophic plant pathogen, without necrosis or dwarfism in the treated plants that would be expected from a fully mutated or nonfunctional SSADH gene, as taught in PCT/US2019/048870. Certainly there are many other plant genes that may be identified as useful for transient silencing for the same purpose, as well as for any other purpose.

### DESCRIPTION OF EXEMPLARY EMBODIMENTS

### Sequences

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the exact RNA equivalent and the term “identity” or “essentially identical” in reference to a DNA sequence includes an RNA sequence meeting these criteria except that thymine nucleotides are replaced with uracil nucleotides. A given first polynucleotide sequence, whether DNA or RNA, further defines the sequence of its exact complement (which can be DNA or RNA), a second polynucleotide that hybridizes perfectly to the first polynucleotide by forming Watson-Crick base-pairs. For DNA: DNA duplexes (hybridized strands), base-pairs are adenine: thymine or guanine: cytosine; for DNA: RNA duplexes, base-pairs are adenine: uracil or guanine: cytosine. Thus, the nucleotide sequence of a blunt-ended double-stranded polynucleotide that is perfectly hybridized (where there is “100% complementarity” between the strands or where the strands are “complementary”) is unambiguously defined by providing the nucleotide sequence of one strand, whether given as DNA or RNA. By “essentially identical” or “essentially complementary” to a target gene or a fragment of a target gene is meant that a polynucleotide strand (or at least one strand of a double-stranded polynucleotide) is designed to hybridize (generally under physiological conditions such as those found in a living plant or animal cell) to a target gene or to a fragment of a target gene or to the transcript of the target gene or the fragment of a target gene; one of skill in the art would understand that such hybridization does not necessarily require 100% sequence identity or complementarity. A first nucleic acid sequence is “operably” connected or “linked” with a second nucleic acid sequence when the first nucleic acid sequence is placed in a functional relationship with the second nucleic acid sequence. For example, a promoter sequence is “operably linked” to a DNA if the promoter provides for transcription or expression of the DNA. Generally, operably linked DNA sequences are contiguous.

The term “polynucleotide” commonly refers to a DNA or RNA molecule containing multiple nucleotides and generally refers both to “oligonucleotides” (a polynucleotide molecule of 18-25 nucleotides in length) and longer polynucleotides of 26 or more nucleotides. Polynucleotides also include molecules containing multiple nucleotides including non-canonical nucleotides or chemically modified nucleotides as commonly practiced in the art; see, e.g., chemical modifications disclosed in the technical manual “RNA Interference (RNAi) and DsRNAs”, 2011 (Integrated DNA Technologies Coralville, Iowa). Generally, polynucleotides as described herein, whether DNA or RNA or both, and whether single- or double-stranded, include at least one segment of 18 or more contiguous nucleotides (or, in the case of double-stranded polynucleotides, at least 18 contiguous base-pairs) that are essentially identical or complementary to a fragment of equivalent size of the DNA of a target gene or the target gene's RNA transcript. Throughout this disclosure, “at least 18 contiguous” means “from about 18 to about 10,000, including every whole number point in between”. Thus, embodiments of this invention include oligonucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

Throughout this disclosure, “at least 18 contiguous” means “from about 18 to about 10,000, including every whole number point in between”. Thus, embodiments of this invention include oligonucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

The polynucleotides described herein can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments include those wherein the polynucleotide is selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used. In some embodiments, the polynucleotide is double-stranded RNA of a length greater than that which is typical of naturally occurring regulatory small RNAs (such as endogenously produced siRNAs and mature miRNAs). In some embodiments, the polynucleotide is double-stranded RNA of at least about 30 contiguous base-pairs in length. In some embodiments, the polynucleotide is double-stranded RNA with a length of between about 50 to about 500 base-pairs. In some embodiments, the polynucleotide can include components other than standard ribonucleotides, e.g., an embodiment is an RNA that comprises terminal deoxyribonucleotides. It will be appreciated that specific sequence information provided herein in the form of DNA sequences includes their RNA corollary sequences.

By “expressing a polynucleotide in the plant” is generally meant “expressing an RNA transcript in the plant”, e.g., expressing in the plant an RNA comprising a ribonucleotide sequence that is anti-sense or essentially complementary to at least a fragment of a target gene or DNA having a sequence selected from the Target Gene Sequences Group, the Trigger Sequences Group, or the DNA complement of any thereof. Embodiments include those in which the polynucleotide expressed in the plant is an RNA comprising at least one segment having a sequence selected from the Trigger Sequences Group, or the complement thereof. However, the polynucleotide expressed in the plant can also be DNA (e.g., a DNA produced in the plant during genome replication), or the RNA encoded by such DNA. Related aspects of the invention include isolated polynucleotides of use in the method and plants having improved Lepidopteran resistance provided by the method.

### Permitted Mismatches

“Essentially identical” or “essentially complementary”, as used herein, means that a polynucleotide (or at least one strand of a double-stranded polynucleotide) has sufficient identity or complementarity to the target gene or to the RNA transcribed from a target gene (e.g., the transcript) to suppress expression of a target gene (e.g., to effect a reduction in levels or activity of the target gene transcript and/or encoded protein). Polynucleotides as described herein need not have 100 percent identity or complementarity to a target gene or sequence or to the RNA transcribed from a target gene to suppress expression of the target gene (e.g., to effect a reduction in levels or activity of the target gene transcript or encoded protein, or to provide control of a Lepidopteran pest). In some embodiments, the polynucleotide or a portion thereof is designed to be essentially identical to, or essentially complementary to, a sequence of at least 18 or 19 contiguous nucleotides in either the target gene or the RNA transcribed from the target gene. In some embodiments, the polynucleotide or a portion thereof is designed to be 100% identical to, or 100% complementary to, one or more sequences of 21 contiguous nucleotides in either the target gene or the RNA transcribed from the target gene. In certain embodiments, an “essentially identical” polynucleotide has 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the endogenous target gene or to an RNA transcribed from the target gene. In certain embodiments, an “essentially complementary” polynucleotide has 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene.

Sequence identity: The term “sequence identity” or “identity,” as used herein in the context of two polynucleotides or polypeptides, refers to the residues in the sequences of the two molecules that are the same when aligned for maximum correspondence over a specified comparison window.

As used herein, the term “percentage of (or percent) sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. The term “about” with respect to a numerical value of a sequence length means the stated value with a +/−variance of up to 1-5 percent. For example, about 30 contiguous nucleotides means a range of 27-33 contiguous nucleotides, or any range in between. The term “about” with respect to a numerical value of percentage of sequence identity means the stated percentage value with a +/−variance of up to 1-3 percent rounded to the nearest integer. For example, about 90% sequence identity means a range of 87-93%. However, the percentage of sequence identity cannot exceed 100 percent. Thus, about 98% sequence identity means a range of 95-100%.

Polynucleotides containing mismatches to the target gene or transcript can be used in certain embodiments of the compositions and methods described herein. In some embodiments, the polynucleotide includes at least 18 or at least 19 or at least 21 contiguous nucleotides that are essentially identical or essentially complementary to a segment of equivalent length in the target gene or target gene's transcript. In certain embodiments, a polynucleotide of 18, 19, 20, or 21 or more contiguous nucleotides that is essentially identical or essentially complementary to a segment of equivalent length in the target gene or target gene's transcript can have 1 or 2 mismatches to the target gene or transcript (i.e., 1 or 2 mismatches between the polynucleotide's 21 contiguous nucleotides and the segment of equivalent length in the target gene or target gene's transcript). In certain embodiments, a polynucleotide of about 50, 100, 150, 200, 250, 300, 350 or more nucleotides that contains a contiguous 18, 19, 20, or 21 or more nucleotide span of identity or complementarity to a segment of equivalent length in the target gene or target gene's transcript can have 1 or 2 or more mismatches to the target gene or transcript.

In designing polynucleotides with mismatches to an endogenous target gene or to an RNA transcribed from the target gene, mismatches of certain types and at certain positions that are more likely to be tolerated can be used. In certain embodiments, mismatches formed between adenine and cytosine or guanosine and uracil residues are used as described by Du et al. (2005) Nucleic Acids Res., 33:1671-1677. In some embodiments, mismatches in 19 base-pair overlap regions are located at the low tolerance positions 5, 7, 8 or 11 (from the 5′ end of a 19-nucleotide target), at medium tolerance positions 3, 4, and 12-17 (from the 5′ end of a 19-nucleotide target), and/or at the high tolerance positions at either end of the region of complementarity, i.e., positions 1, 2, 18, and 19 (from the 5′ end of a 19-nucleotide target) as described by Du et al. (2005) Nucleic Acids Res., 33:1671-1677.

In some embodiments, the present invention teaches more effective down-regulation of a plant target gene SSADH, homolog or ortholog, in which said homolog or ortholog shares at least 99.9%, 99.8%, 99.7%, 99.6%, 99.5%, 99.4%, 99.3%, 99.2%, 99.1%, 99%, 98.9%, 98.8%, 98.7%, 98.6%, 98.5%, 98.4%, 98.3%, 98.2%, 98.1%, 98%, 97.9%, 97.8%, 97.7%, 97.6%, 97.5%, 97.4%, 97.3%, 97.2%, 97.1%, 97%, 96.9%, 96.8%, 96.7%, 96.6%, 96.5%, 96.4%, 96.3%, 96.2%, 96.1%, 96%, 95.9%, 95.8%, 95.7%, 95.6%, 95.5%, 95.4%, 95.3%, 95.2%, 95.1%, 95%, 94.9%, 94.8%, 94.7%, 94.6%, 94.5%, 94.4%, 94.3%, 94.2%, 94.1%, 94%, 93.9%, 93.8%, 93.7%, 93.6%, 93.5%, 93.4%, 93.3%, 93.2%, 93.1%, 93%, 92.9%, 92.8%, 92.7%, 92.6%, 92.5%, 92.4%, 92.3%, 92.2%, 92.1%, 92%, 91.9%, 91.8%, 91.7%, 91.6%, 91.5%, 91.4%, 91.3%, 91.2%, 91.1%, 91%, 90.9%, 90.8%, 90.7%, 90.6%, 90.5%, 90.4%, 90.3%, 90.2%, 90.1%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, or 40% sequence identity to SSADH.

### Recombinant DNA Constructs

Another aspect of this invention provides a recombinant DNA nucleic acid construct encoding a SAM RdRp operably linked to an RNA subgenome including at least one segment of 15, 16, 17, 18, 19, 20, 21, 22, 23, 24, or 25 more contiguous nucleotides with a sequence of about 70% to about 100% identity with a segment of equivalent length of an RNA capable of forming a dsRNA, including, but not limited to, DNA having a sequence selected from the group consisting of SEQ ID NO:4 The recombinant nucleic acid constructs are useful in providing a plant having improved resistance to bacterial or fungal infections, e.g., by expressing in a plant an RNA subgenome of such a recombinant nucleic acid construct. The contiguous nucleotides can number more than 15, 16, 17, 18, 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, or greater than 900 contiguous nucleotides, as for example, from SEQ ID NO:1.

The contiguous nucleotides can number more than about 900, about 1000, about 1100, about 1200, about 1300, about 1400, about 1500, about 1600, about 1700, about 1800, about 1900, about 2000, about 2100, about 2200, about 2300, about 2400, about 2500 contiguous nucleotides, as for example, from SEQ ID NO:1.

In some embodiments, the recombinant nucleic acid constructs of this invention are provided in a recombinant vector. By “recombinant vector” is meant a recombinant polynucleotide molecule that is used to transfer genetic information from one cell to another. Embodiments suitable to this invention include, but are not limited to, recombinant plasmids, recombinant cosmids, artificial chromosomes, and recombinant viral vectors such as recombinant plant virus vectors, including RNA viruses and recombinant baculovirus vectors. Typically, nonrecombinant would relate to sequences that are wholly synthesized.

### RNA Interference

Sequence-selective, post-transcriptional inactivation of expression of a target gene can be achieved in a wide variety of eukaryotes by introducing double-stranded RNA (dsRNA) corresponding to the target gene, a phenomenon termed RNA interference (RNAi). RNAi occurs when an organism recognizes dsRNA molecules and hydrolyzes them. The resulting hydrolysis products are small RNA fragments of 15, 16, 17, 18, 19, 20, 21, 22, 23 or 24 nucleotides in length, called small interfering RNAs (siRNAs) or microRNAs (miRNAs). The siRNAs then diffuse or are carried throughout the organism, including across cellular membranes, where they hybridize to mRNAs (or other RNAs) and cause hydrolysis of the RNA. Most plant miRNAs show extensive base pairing to, and guide cleavage of their target mRNAs (Jones-Rhoades et al. (2006) Annu. Rev. Plant Biol. 57, 19-53; Llave et al. (2002) Proc. Natl. Acad. Sci. USA 97, 13401-10406). In other instances, interfering RNAs may bind to target RNA molecules having imperfect complementarity, causing translational repression without mRNA degradation.

The term “RNAi” or “RNA interference” refers to the process of sequence-specific post-transcriptional gene silencing (e.g., in nematodes), mediated by double-stranded RNA (dsRNA). “dsRNA” refers to RNA that is partially or completely double stranded. Antisense RNA that binds to an mRNA transcript forms dsRNA. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism (e.g., plants and/or crops), by, e.g., transformation, injection, spray, brush, mechanical abrasion, laser etching or immersion, etc. After introduction into the organism, the target gene-specific dsRNA is processed into relatively small fragments (siRNAs) and can subsequently become rapidly distributed long distance throughout an entire large plant, including commercially grown citrus trees, leading to a loss-of-function mutation having a phenotype that, over the period of a generation, may come to closely resemble the phenotype arising from a complete or partial deletion of the target gene.

This approach takes advantage of the discovery that siRNA can trigger the degradation of mRNA corresponding to the siRNA sequence. RNAi is a remarkably efficient process whereby dsRNA induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490).

The effects of RNAi can be both systemic and heritable in plants. In plants, RNAi is thought to propagate by the transfer of siRNAs between cells through plasmodesmata. The heritability comes from methylation of promoters targeted by RNAi; the new methylation pattern is copied in each new generation of the cell. A broad general distinction between plants and animals lies in the targeting of endogenously produced miRNAs; in plants, miRNAs are usually perfectly or nearly perfectly complementary to their target genes and induce direct mRNA cleavage by RISC, while animals' miRNAs tend to be more divergent in sequence and induce translational repression. Detailed methods for RNAi in plants are described in David Allis et al (Epigenetics, CSHL Press, 2007, ISBN 0879697245, 9780879697242), Sohail et al (Gene silencing by RNA interference: technology and application, CRC Press, 2005, ISBN 0849321417, 9780849321412), Engelke et al. (RAN Interference, Academic Press, 2005, ISBN 0121827976, 9780121827977), and Doran et al. (RNA Interference: Methods for Plants and Animals, CABI, 2009, ISBN 1845934105, 9781845934101), which are all herein incorporated by reference in their entireties for all purposes.

The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (i.e., a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule.

dsRNA-mediated regulation of gene expression in plants is well known to those skilled in the art. See, e.g., WIPO Patent Application Nos. WO1999/061631A and WO1999/053050A, each of which is incorporated by reference herein in its entirety.

In some embodiments, an RNAi agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs (siRNAs) with characteristic two base 3′ overhangs (Bernstein, et al., (2001) Nature 409:363). In plants, these siRNAs, which are double stranded, rapidly become phloem mobile. The siRNAs can incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188).

Chimeric dsRNA.

The dsRNA can be comprised of 21-mers selected by computer programs predicting where dicing will occur in a target gene, so that rather than incorporate, say 210 bp of a contiguous target gene region, chimeras can be synthesized that have 21 mers predicted to be good silencing candidates. If these are combined together as a chimera they may work much better (10×21-mer) than a single contiguous 210 bp region. Furthermore, chimeras need not be limited to 21-mers from a single gene, but can include “stacked” targets of 2, 3 or more genes together. In some embodiments, the present invention anticipates the use of RNA interference (RNAi) for the down-regulation of multiple target genes, or homologs or orthologs of multiple genes. Thus in some embodiments the present invention teaches the expression of antisense, inverted repeat, small RNAs, artificial miRNA, or other RNAi triggering sequences.

### GVA RdRP, a Self-Amplifying RNA (SAM)

It is well understood that positive strand RNA alphaviruses can be used to create self-amplifying vaccines for animals, which possess a circulatory system (Geall et al, 2012; Lou et al., 2020). Plants have no circulatory system. There is no teaching or suggestion that a plant alphavirus could be used to create dsRNA in plants for the purpose of silencing one or more plant genes rapidly and over long distances. In some embodiments the present invention also teaches expression vectors capable of producing inhibitor nucleic acid molecules, particularly the mRNA encoding the RNA-dependent RNA polymerase (RdRp), of grapevine virus A (GVA) (Galiakparov et al., 2003) which could drive expression of a subgenome comprised of sense and antisense RNA matching any plant or insect gene of interest for the purposes of silencing. The subgenome could form a hairpin RNA that forms a dsRNA. GVA is a Vitivirus with a host range limited to grapes (du Preez et al., 2011; Galiakparov et al., 1999). There is no teaching or suggestion that the RdRp from GVA can function in any plant cell outside of grapevine, tobacco and Chenopodium spp. (du Preez et al., 2011).

In some embodiments the RNAi constructs of the present invention comprise sequences capable of triggering RNAi suppression of SSADH genes, including nucleic acid fragments comprising sequence identities higher than about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% to one or more gene targets or regions of the same gene.

In some embodiments, the antisense or small RNA molecules are targeted to a section of the coding portion of the target gene. In other embodiments, the RNAi sequences of the present invention are targeted to the 5′ or 3′ untranslated regions (UTRs) of the target gene. In yet other embodiments, the RNAi sequences of the present invention are targeted to the promoter of the target gene. Methods of selecting sequence target regions for RNAi molecule design are described in more detail in (Fougerolles, et al., 2007; U.S. Pat. No. 7,732,593).

In some embodiments, the SAM molecules of the invention may be modified at various locations, including the sugar moiety, the phosphodiester linkage, and/or the base. For example, in order to further increase the stability of the molecules in vivo, the 3′-end of the hairpin structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety (U.S. Patent Publication 2011/251258).

In some embodiments, the present invention also teaches the down-regulation of genes via antisense technology. In some embodiments, the present invention can be practiced using other known methods for down-regulating gene expression including T-DNA knockout lines, tilling, TAL-mediated gene disruption, transcriptional gene silencing, and site-directed methylations.

### Application of RNAi Formulation/Treatment

Plant recombinant technology is one vehicle for delivering gene silencing of target genes, either endogenous plant target genes or target genes of a plant pest organism. In general, a plant is transformed with DNA that is incorporated into the plant genome, and when expressed produces a dsRNA that is complementary to a gene of interest, which can be an endogenous plant gene or an essential gene of a plant pest. Plant recombination techniques to generate transgene and beneficial plant traits require significant investments in research and development, and pose significant regulatory hurdles. Methods and formulations for delivering dsRNA into plant cells by exogenous application to exterior portions of the plant, such as leaf, stem, and/or root surfaces for regulation of endogenous gene expression are known in the art. See, e.g., U.S. Pat. No. 9,433,217, U.S. Patent Publication 2013/0047298, Chinese Patent No. 103748230B and Chinese Patent Publication CN101914540A, each of which is incorporated by reference herein in its entirety. Such methods and formulations represent a significant development for gene silencing technology using RNAi, which has significantly fewer regulatory hurdles. SAM technology applied to plants to form dsRNAs acting rapidly and over long distances are similarly significant and with fewer regulatory hurdles.

In some embodiments, the present invention teaches methods and formulations to topically apply exogenous RNA molecules to external tissue surfaces of plants. In some embodiments, the application exogenous RNA molecules, including mRNA, dsRNA, siRNA, micro-RNA (miRNA) and antisense RNA (aRNA), causes silencing of plant endogenous target genes or of the target genes of plant pests in the plant cells nearby the external tissue surfaces. In some embodiments, the application exogenous RNA molecules, including mRNA, dsRNA, siRNA, miRNA and aRNA, causes silencing of plant endogenous target genes or of the target genes of plant pests in the plant cells that are located at a long distance from the external tissue surfaces to which they are applied.

In some embodiments, the present invention provides that applying a SAM formulation (and/or treatments) by spray, brush, mechanical abrasion, laser etching or immersion application of SAM molecules, or other non-tissue invasive techniques, leads to absorption and assimilation of the exogenous RNA molecules into nearby or distant plant cells, thus causing long distance endogenous and/or pest gene silencing.

In some embodiments, the present invention teaches methods of repressing, preventing, eliminating, reducing, or otherwise ameliorating a bacterial or fungal infection of a plant comprising topical application of nucleic acid including DNA molecules as well as RNA molecules including mRNA, dsRNA, siRNA, miRNA and aRNA.

### Example 1: GV-RNA-CSEeGFPv2 RdRp Expressed Subgenomic GFP in Tobacco, and Self-Amplified

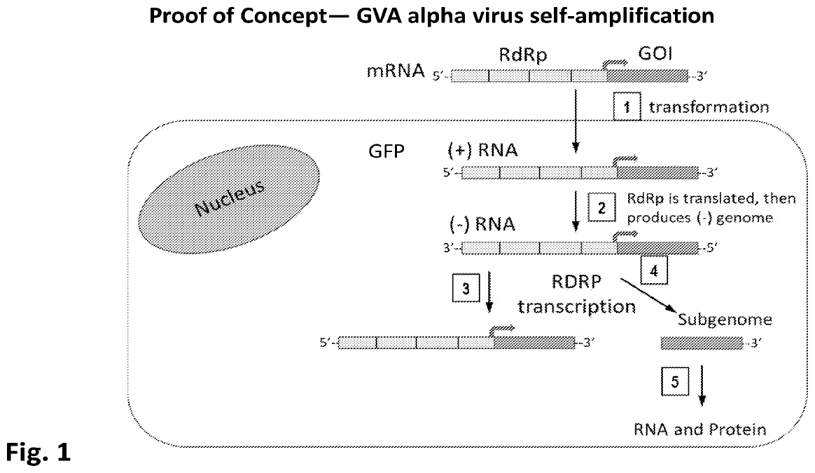

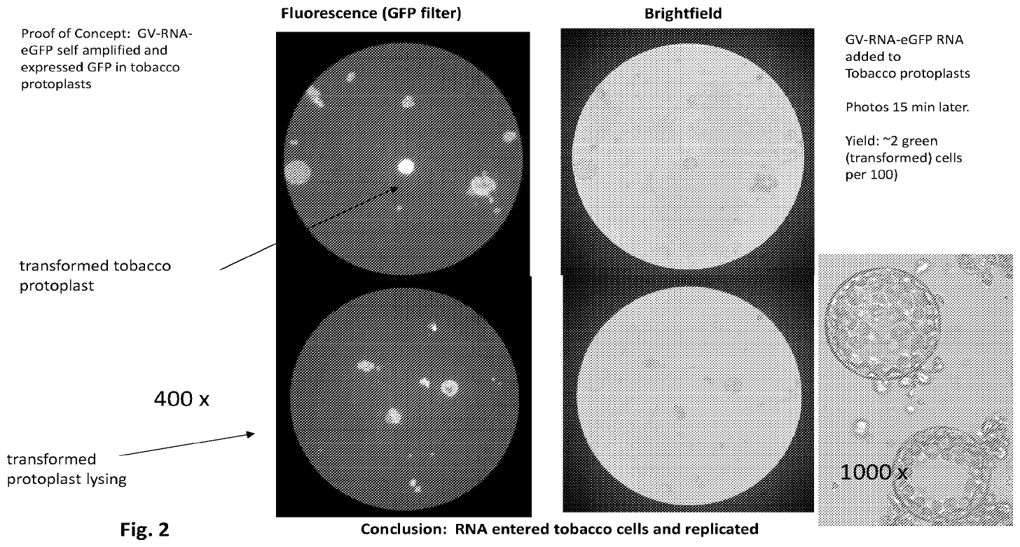

Many eukaryotic viruses encode RdRps that, along with certain replaceable subgenomic regions are operationally linked and self-amplifying (SAM), including grapevine virus A (GVA), NCBI Reference Sequence: NC_003604.2. We had the genes encoding GVA RdRp and a subgenomic region encoding Green Fluorescent Protein (GFP) synthesized commercially (SEQ ID NO: 5; FIG. 1). For proof of concept, the SAM mRNA, including the RdRp and subgenomic region was expressed in vitro, capped, and the SAM mRNA was transformed into tobacco protoplasts. The GFP protein was highly expressed from the subgenomic region operationally linked to the GVA RdRp in nonhost tobacco protoplasts (FIG. 2). This demonstrated that the RdRp from GVA could amplify RNA in plant cells other than grapevine, confirming replication of this SAM in tobacco. From 1-2% of the protoplasts glowed very brightly from the added RNA, indicating both self-amplification of the mRNA and good gene expression in tobacco.

### Example 2. DsRNA Chimeras Synthesized and Applied as a Foliar Spray Using Laser Etching to Heavily Infected Grapefruits in a Commercial Citrus Grove Significantly, but Transiently Suppressed the Citrus Target Gene

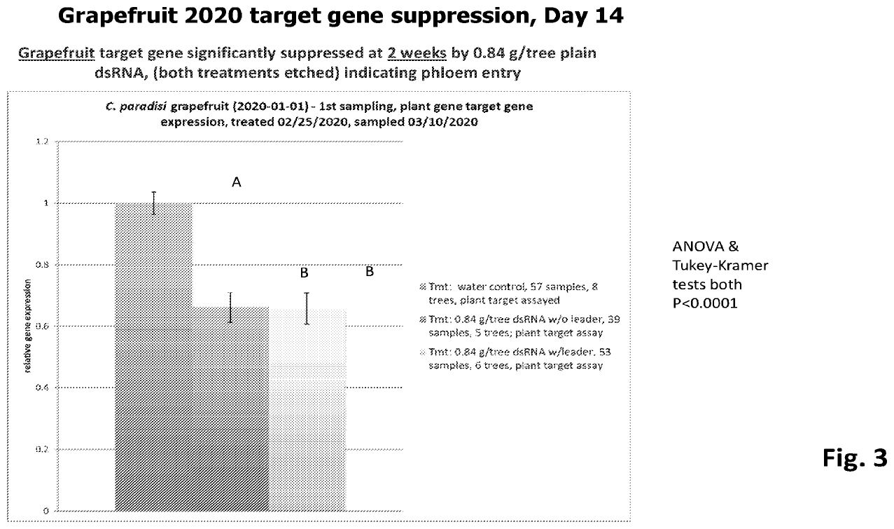

The original dsRNAs used to treat heavily infected citrus in commercial Hamlins and grapefruits and disclosed in PCT/US2019/048870 were randomly selected large (300-500 bp) blocks of the target gene SSADH mRNA coding or regions after removal of exon sequences, and these were synthesized commercially and directly applied to citrus using laser etching followed by spraying an aqueous solution of the dsRNAs. In order to improve upon the use of randomly selected large random blocks of native dsRNA for siRNA production, we also designed large (300-500 bp) synthetic chimeras consisting of small (21 bp) blocks of sequence selected from various exon regions of SSADH using siRNA-Finder (Si-Fi) software (Luck et al., 2019), forming the “IPG-1” and “IPG-2” chimeras. These larger sized dsRNA chimeras were synthesized commercially in large quantities and directly applied at a high rate of 0.84 g/tree to 48 large trees (12 year old) in a commercial grove, again using laser etching. Ninety two samples were taken from all around the perimeters of each of 11 of the trees, including unsprayed regions, and these samples together showed significant reduction in the expression of the plant target gene SSADH two weeks after dsRNA application, compared to 57 samples taken from water treated controls (FIG. 3). No significant reduction in the target gene was observed one week or 4 weeks after treatment with the chimeric dsRNA. This demonstrated that large amounts (nearly 1 g per tree) of a water suspension of dsRNA applied as a foliar spray with laser etching can transiently suppress a citrus target gene in all assayed parts of the tree, including parts that were untreated. The suppression of the target gene in leaves that had been untreated indicated that the chimeras were diced to produce siRNA that became systemic.

### Example 3. Commercially Synthesized dsRNA Chimeras Encapsulated into Nanoemulsions and Applied to a Single Branch on Field Grown Lemons were Diced to Form siRNA that Moved Long Distances in Field Grown Citrus Trees

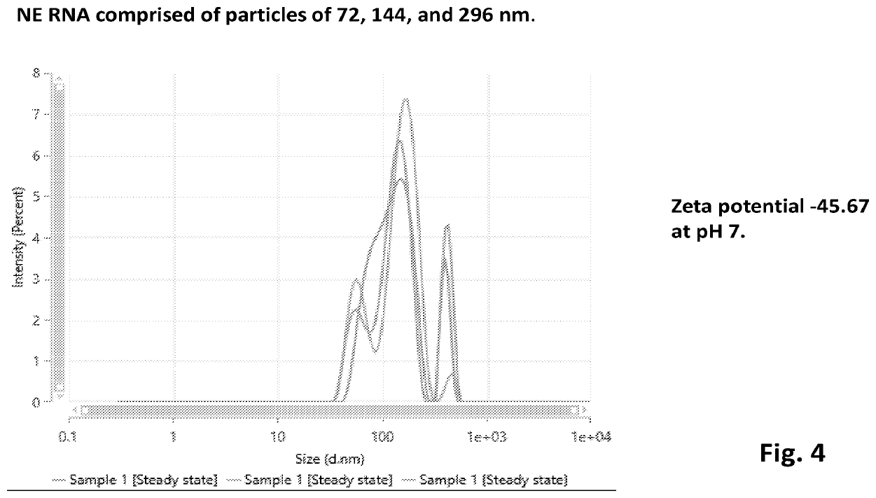

To attempt to facilitate uptake of the extremely hydrophilic and anionic dsRNA into plant cells, the long (ca. 500 bp) synthesized SSADH chimeras IPG-1 and IPG-2 were formulated into nanoemulsions (NEs) comprised primarily of lecithin and gelatin, using general methods disclosed by Cui et al. (2005) and modified by Pérez et al. (2012). The resulting particles ranged in size from 72 to 296 nm, with a major peak of 144 nm (FIG. 4). with a Zeta potential-45.67 at pH 7. These dsRNA, lecithin coated nanoemulsions become positively charged at acidic pH (Pérez et al., 2012); citrus phloem has an acidic pH of 6.0 (Hijaz & Killiny 2014).

These nanoemulsions carrying long chimeric dsRNAs were applied to field grown lemons on a single labeled branch of each tree by hand spraying, taking care not to overspray and to protect uninoculated branches from exposure to the dsRNA solution. RNA was extracted from the leaves of the treated plants RNA samples were taken from each sprayed branch, and from each of the five other untreated branches around each tree. The untreated branches were evenly distributed around the trees. A total of six labeled branches (1 treated and 5 untreated) around each of 12 different trees were sampled per dsRNA treatment. Samples were taken on Days 1, 7 and 14 after inoculation and from the same branches. There were six untreated control trees, sampled on Days 1 and 7. For all samples, crude RNA was extracted, treated with DNase and then cDNA was synthesized based on the stem-loop RT-PCR protocol exactly as described by Chen et al (2005) and Varkonyi (2007). Four sets of stem loop RT primers were designed to amplify 4 different 21 mer sequences predicted after dicing and common to both of the synthesized chimeras. One of these four primer sets proved to be far more robust than the others and was used in all subsequent sample testing.

The 21 mer sequence common to both IPG-1 and IPG-2 was not detected in water control trees on any branch tested, but was detected on 7/72 (10%) of untreated tree branches on Day 1 after spraying with a plain water solution of IPG-1, and also on 17/72 (24%) untreated tree branches on Day 1 when using IPG-1 coated with lecithin in a nanoemulsion. A total of 144 samples were taken each sampling period, 72 from each treatment. This demonstrated movement of presumably diced IPG-1 siRNA from treated branches to untreated branches. A total of 36 water control samples were taken on Day 1. The same trees and labeled branches were sampled on Days 7 and 14; all data are summarized in Table 1 below. Movement to untreated branches maximized at 7 days and began fading by 14 days following treatment. This further confirmed dicing of the chimeric dsRNA to form siRNA. Significantly better movement of siRNA was detected by stem loop PCR when citrus was treated with long dsRNA encapsulated in a lecithin nanoemulsion than with the same dsRNA was applied as dsRNA applied in plain water. These results indicated that long dsRNA gains access to phloem cells (where dicing occurs) more efficiently when it is encapsulated in a lecithin nanoemulsion.

TABLE 1
Number of samples from treated and untreated branches testing
positive for siRNA using stem loop PCR out of total samples
tested. Percentage testing positive in parentheses ( ).

| | Water | Plain dsRNA | | Lecithin emulsed |
|---|---|---|---|---|
| | controls | in water | | dsRNA in water |
| Day 1 (untreated only) | 0/30 (0%) | 7/60 | (12%) | 17/60 (28%) |
| (all branches, including | 0/36 (0%) | 12/72 | (17%) | 26/72 (36%) |
| treated) | | | | |
| Day 7 (untreated only) | 0/30 (0%) | 11/60 | (18%) | 41/60 (68%) |
| (all branches, including | 0/36 (0%) | 16/72 | (22%) | 52/72 (72%) |
| treated) | | | | |
| Day 14 (untreated only) | ND | 4/60 | (7%) | 23/60 (38%) |
| (all branches, including | | 7/72 | (10%) | 29/72 (40%) |
| treated) | | | | |

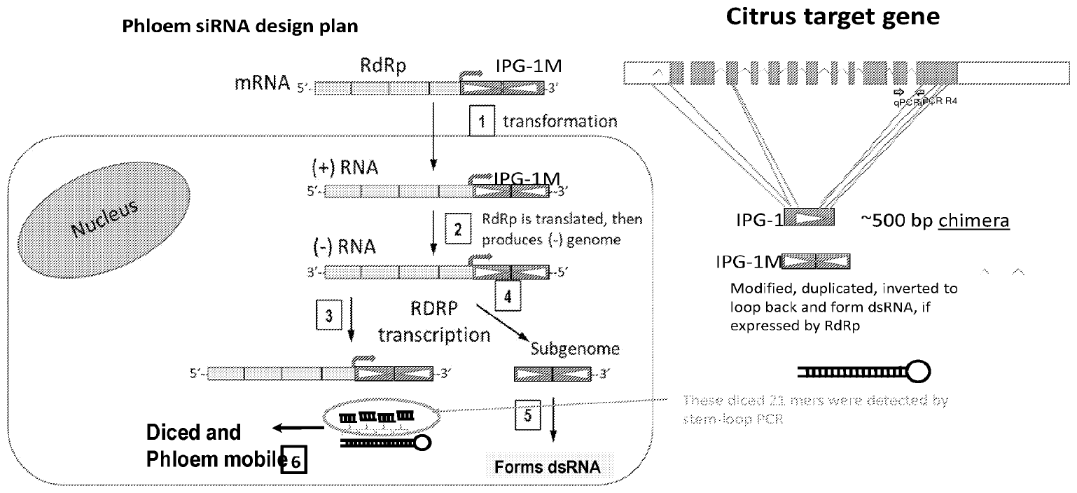

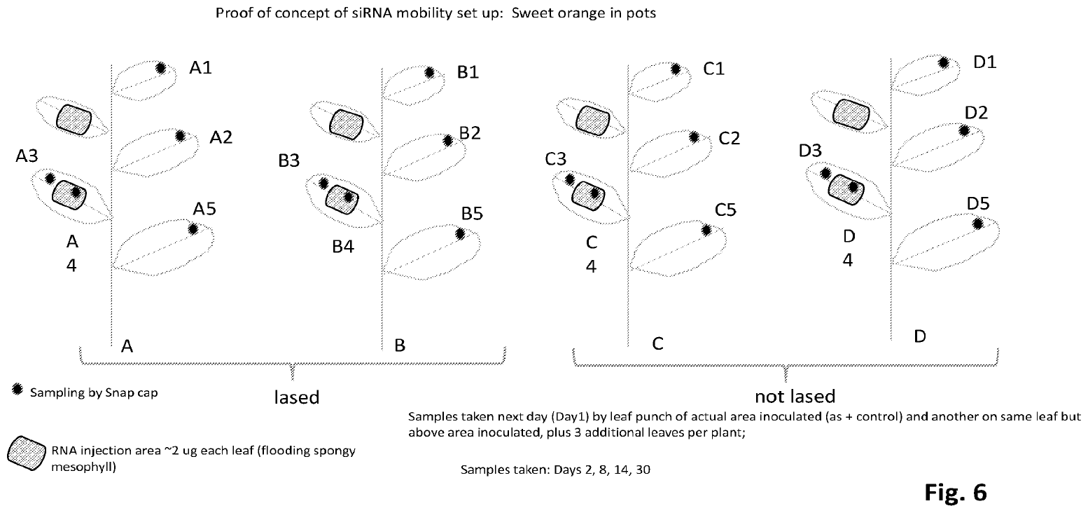

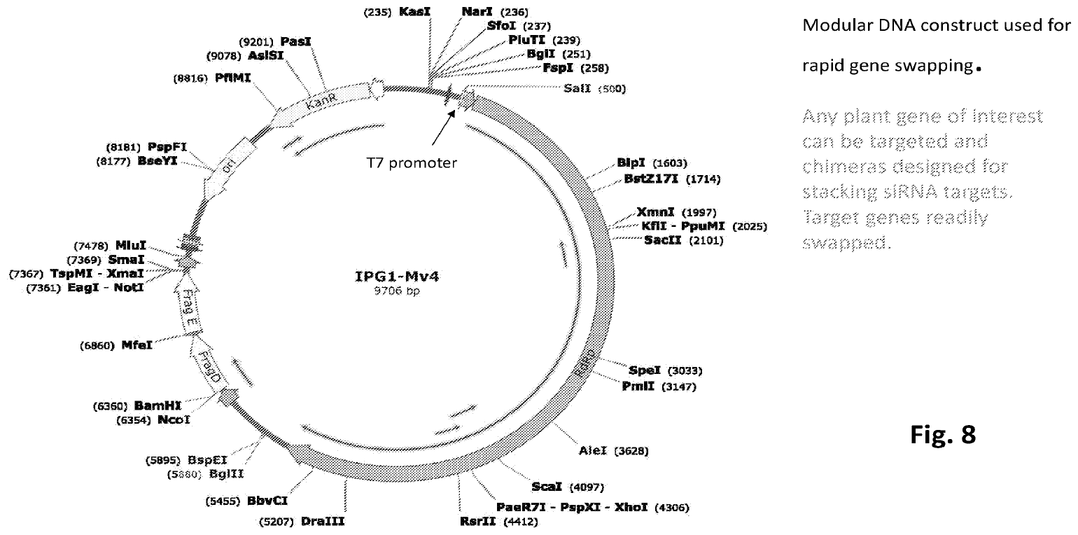

Example 4. Synthesis of a SAM expressing a subgenomic dsRNA chimera that forms siRNA and moves long distances. We then replaced the subgenomic GFP of the SAM illustrated in Example 1 with sense and antisense strands based on the dsRNA chimera IPG-1 known to cause RNAi (FIG. 3) and illustrated in Example 2. Chimeras that were predicted to result in siRNAs were designed based on various exon regions of SSADH, although one or more citrus target genes could be used. The antisense sequence to IPG-1 was also synthesized, along with a 15 bp loop, and the sequence was modified to eliminate any likely ribosome binding sites to prevent protein translations, resulting in IPG-1M. This construct was commercially synthesized and swapped with the GFP SAM construct illustrated in FIG. 2 (GV-RNA-eGFP), forming IPG1-Mv4 (SEQ ID NO. 2 and FIG. 8). This created a SAM that formed dsRNA from the subgenome of the expressed mRNA that could be diced and become phloem mobile (FIG. 5). The dsRNA regions of these SAMs were chimeric genes developed from sequences taught in earlier patent applications (ie, PCT/US2015/062698 and PCT/US2019/048870) to have some disease control effect in commercial citrus field trials due to siRNA silencing of citrus host genes. The DNA sequence of the resulting SAM that formed dsRNA as shown in FIG. 5 is provided as SEQ ID NO. 2. In vitro transcription reactions were performed to create over 600 mcg of mRNA, and the mRNAs were capped, diluted to 800 mcl in water, incorporated onto gelatin and lecithin nanoemulsions as described in Example 3 for dsRNA and used to inoculate 8 labeled citrus leaves, two on each of four small potted sweet orange trees, each about 1.5′ tall above soil line by syringe injection (approximately 140 mcg/tree). An additional 3 uninoculated citrus leaves on each plant were also labeled for sampling purposes; one from a branch above (Apex Samples A1, B1, C1, D1); one from a branch in between (Middle samples A2, B2, C2, D2), and one below the branches (Low samples A5, B5, C5, D5) carrying the attached inoculated leaves, which were sampled outside (A3, B3, C3, D3) and inside (A4, B4, C4, D4) the inoculation zone, which was marked by ink on the leaf. Approximately 70 mcg of mRNA was syringe inoculated by flooding the spongy mesophyll in two areas across the midvein, in each of the two leaves inoculated, as illustrated in FIG. 6. This resulted in each tree being inoculated with a total of 140 mcg of capped, SAM mRNA in a nanoemulsion.

Samples were taken from each labeled leaf on Days 2, 8, 14 and 30 after syringe inoculation of the dsRNA encoding the SAM that was designed to express dsRNA that could be diced. Results that siRNA was produced and was phloem mobile are presented in Table 2 below:

TABLE 2
Number of positive samples by stem loop PCR to detect siRNA from
sweet orange plants with labeled, uninjected leaves (apex, middle
and low) and uninjected and injected zones of leaves injected with
mRNA encoding a SAM designed to make siRNA and testing positive
for siRNA using stem loop PCR out of total samples tested.

| | SAM Injected leaves | | | | |
|---|---|---|---|---|---|
| | Inside | Outside | Uninjected leaves of SAM | | |
| | injection | injection | treated plants | | |
| | zone | zone | Apex | Middle | Low |
| Day 2 | ¾ | ¼ | ¼ | 0 | ¼ |
| Day 8 | 2/4 | 2/4 | ¾ | ¾ | 4/4 |
| Day 15 | ¾ | 0 | 0 | ¼ | 0 |
| Day 30 | 0 | ¼ | 0 | 0 | 0 |

Clearly, siRNA was made, moved and was detected by Day 2, maximized by Day 8, declined by Day 15 and nearly disappeared by Day 30. These results are consistent with the results presented in Example 3 using commercially synthesized dsRNA, except that nearly a gram (840,000 mcg) of dsRNA was applied to field grown trees in Example 2 and Example 3, compared to 140 mcg of SAM mRNA applied in this example, a reduction on the order of 6,000×. This indicated that the SAM mRNA expressed the encoded dsRNA abundantly in phloem, where it was diced to produce siRNA, which became phloem mobile and was detected by Day 8. The 6,000× reduction in the amount of RNA needed for application indicated that the SAM concept can be usefully applied on an agricultural scale to suppress targeted citrus genes.

### Example 5. SAM mRNA Systemically Suppressed of the Expression of the Citrus Target Gene SSADH Similarly to Synthetic dsRNA Chimeras Applied as a Foliar Sprays

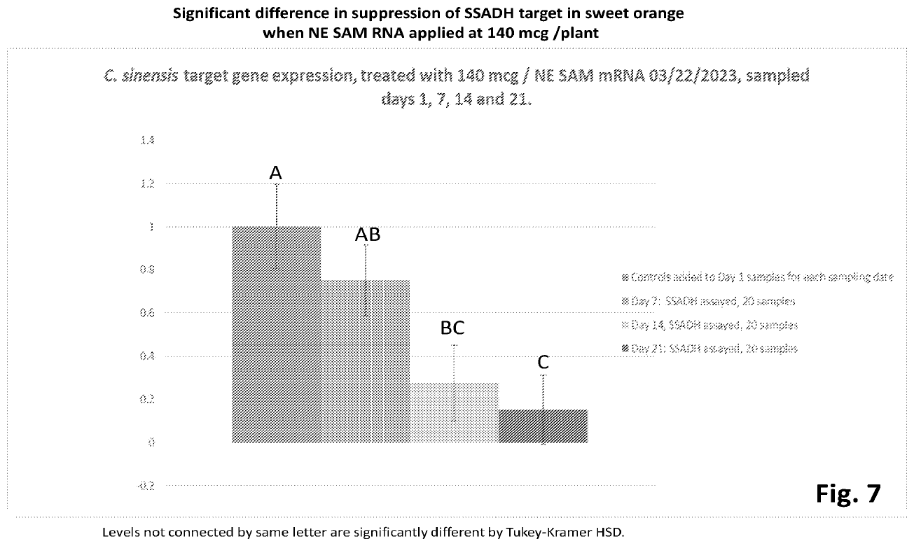

In order to determine if the IPG1-Mv4 DNA construct could be used to suppress expression of the citrus target SSADH gene, the DNA construct was linearized with Mlul, expressed in vitro using MegaScript T7 polymerase (Thermo Fisher Scientific), to produce over a milligram of SAM mRNA that was then capped using the Vaccinia virus capping enzyme (New England Biolabs). The capped SAM mRNA was encapsulated into a freshly made nanoemulsion and syringe injected into citrus leaves as indicated in FIG. 6. The next day, RNA samples from both inoculated and uninoculated leaves were extracted for real time reverse transcriptase qPCR (qRT-PCR) analysis to quantify levels of SSADH expression over time compared to Day 1 controls. Plant elongation factor 1a (EF1a) was used as a comparator to normalize the relative abundance of the target gene in each sample. Results are presented in FIG. 7, showing that the citrus target gene SSADH was suppressed by Day 7, significantly suppressed by Day 14 to less than 30% of normal levels and significantly suppressed by Day 21 to less than 20% of normal levels. Combined with the demonstration that 6,000× less SAM RNA could be applied to suppress a targeted citrus gene (both in grapefruit and sweet orange) is a clear demonstration of potential agricultural utility in plants.

Combined with the demonstration of expression of SAM mRNA in tobacco plants and the fact that the source of the SAM polymerase system is grapevine indicates that this system could be applied to silence any plant gene, not just those in citrus.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

All publications, patents, patent publications, and nucleic acid and amino acid sequences cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### CITED REFERENCES

de Andrade, E. C., and Hunter, W. B. 2016. RNA Interference-Natural Gene-Based Technology for Highly Specific Pest Control (HiSPeC). http://dx.doi.org/10.5772/61612
Brunings, A. M. and Gabriel, D. W. 2003. Xanthomonas citri: breaking the surface. Molecular Plant Pathology 4:141-157.
Chen C, Ridzon D A, Broomer A J, Zhou Z, Lee D H, Nguyen J T, Barbisin M, Xu N L, Mahuvakar V R, Andersen M R, Lao K Q, Livak K J, Guegler K J. 2005. Real-time quantification of microRNAs by stem-loop RT-PCR. Nucleic Acids Res. 33: e179. doi: 10.1093/nar/gni178.
Chibucos, M. C., C. W. Collmer, T. Torto-Alalibo, M. Gwinn-Giglio, M., Lindeberg, D. Li, and B. M. Tyler. 2009. Programmed cell death in host symbiont associations, viewed through the Gene Ontology. BMC Microbiol. 9 (Suppl. 1): S5.29.
Cui, Z., Qiu, F., and B. R. Sloat. 2006. Lecithin-based cationic nanoparticles as a potential DNA delivery system,” Int. J. of Pharmaceutics 313:206-213.
Deng, J.-X., Nie, X.-J., Lei, Y.-F., Ma, C.-F., Xu, D.-L., Li, B. and Zhang, G.-C. 2012. The highly conserved 5′ untranslated region as an effective target towards the inhibition of Enterovirus 71 replication by unmodified and appropriate 2′-modified siRNAs. Journal of Biomedical Science, 19 (1), 73. http://doi.org/10.1186/1423-0127-19-73
Dickman, M. B., Park, Y. K., Oltersdorf #, T., Li, W., Clemente, T., French, R. 2001. Abrogation of disease development in plants expressing animal antiapoptotic genes. Proc. Natl. Acad. Sci. USA 98:6957-6962.
Doukhanina, E. V., S. Chen, E. van der Zalm, A. Godzik, J. Reed, and M. B. Dickman. 2006. Identification and functional characterization of the BAG protein family in Arabidopsis thaliana. Journal of Biological Chemistry 281:18793-18801
du Preez, J., Stephan, D., Mawassi, M. & Burger, J. 2011. The grapevine-infecting vitiviruses, with particular reference to grapevine virus A. Archives of virology. 156. 1495-503. 10.1007/s00705-011-1071-3.
Fittipaldi, M., Nocker, A., Codony, F., 2012. Progress in understanding preferential detection of live cells using viability dyes in combination with DNA amplification.
J. Microbiol. Methods. 91 (2), 276-289.
Galiakparov N, Tanne E, Sela I, Gafny R. 1999. Infectious RNA transcripts from grapevine virus A cDNA clone. Virus Genes 19 (3): 235-42. doi: 10.1023/a: 1008192831883. PMID: 10595415.
Galiakparov, N., Tanne, E., Sela, I. & Gafny, R. 2003. Functional analysis of the Grapevine virus A genome. Virology. 306. 42-50. 10.1016/S0042-6822 (02) 00019-3.
Gardner, C. L., Pagliai, F. A., Pan, L., Bojilova, L., Torino, M. I., Lorca, G. L., & Gonzalez, C. F. (2016). Drug Repurposing: Tolfenamic Acid Inactivates PrbP, a Transcriptional Accessory Protein in Liberibacter asiaticus. Frontiers in Microbiology, 7, 1630. http://doi.org/10.3389/fmicb.2016.01630.
Geall, A., Verma, A., & Otten, G. et al. 2012. Nonviral delivery of self-amplifying RNA vaccines. Proc. Nat. Acad. Sci, USA. 109:14604-9. 10.1073/pnas. 1209367109.
Gilchrist, D. and Lincoln, J. 2011. Pierce's Disease control and bacterial population dynamics in winegrape varieties grafted to rootstocks expressing anti-apoptotic sequences. Proceedings of the 2011 Pierce's Disease Research Symposium, Sacramento, pp 118-121.
Hijaz F, Killiny N. 2014. Collection and chemical composition of phloem sap from Citrus sinensis L. Osbeck (sweet orange). PLOS One. 9: e101830. doi: 10.1371/journal.pone.0101830.
Jain M, Cai L, Black I, Azadi P, Carlson R W, Jones K M, Gabriel D W. 2022. ‘Candidatus Liberibacter asiaticus’-Encoded BCP Peroxiredoxin Suppresses Lipopolysaccharide-Mediated Defense Signaling and Nitrosative Stress In Planta. Mol Plant Microbe Interact. 35:257-273. doi: 10.1094/MPMI-09-21-0230-R.
Jain, M., Munoz-Bodnar, A., Zhang, S. and Gabriel, D. W., 2018. A Secreted ‘Candidatus Liberibacter asiaticus’ Peroxiredoxin Simultaneously Suppresses Both Localized and Systemic Innate Immune Responses in Planta. Molecular Plant-Microbe Interactions, https://doi.org/10.1094/MPMI-03-18-0068-R.
Jiang, Y., Ding, X., Chen, X., Wang, Y., Song, W. 2013. The rice XA21 binding protein 25 encodes an ankyrin repeat containing protein and is required for full Xa21-mediated disease resistance. Plant J. 73:814-823.
Kasai A, Bai S, Li T, Harada T (2011) Graft-Transmitted siRNA Signal from the Root Induces Visual Manifestation of Endogenous Post-Transcriptional Gene Silencing in the Scion. PLOS ONE 6 (2): e16895.
Kim, D. Y., Atasheva, S., McAuley, A. J., Plante, J. A., Frolova, E. I., Beasley, D. W. and Frolov, I., 2014. Enhancement of protein expression by alphavirus replicons by designing self-replicating subgenomic RNAs. Proceedings of the National Academy of Sciences, 111 (29), pp. 10708-10713.
Lai C F, Chen C Y & Au L C (2013): Comparison between the repression potency of siRNA targeting the coding region and the 3′-untranslated region of mRNA. Biomed Res Int 2013:637850.
Li Z T, Dhekney S A, Gray D J. 2011. PR-1 gene family of grapevine: a uniquely duplicated PR-1 gene from a Vitis interspecific hybrid confers high-level resistance to bacterial disease in transgenic tobacco. Plant Cell Rep. 30:1-11.
Lacomme, C. and S. S. Cruz. 1999. Bax-induced cell death in tobacco is similar to the hypersensitive response. Proceedings of the National Academy of Sciences of the United States of America 96, no. 14:7956-7961.
Lopes, S. A., Bertolini, E., Frare, G. F., Martins, E. C., Wulff, N. A., Teixeira, D. C., Fernandes, N. G., and Cambra, M. 2009. Graft transmission efficiencies and multiplication of ‘Candidatus Liberibacter americanus’ and ‘Ca. Liberibacter asiaticus’ in citrus plants. Phytopathology 99:301-306.
Lou, G., Anderluzzi, G., Tandrup S., et al. 2020. Delivery of self-amplifying mRNA vaccines by cationic lipid nanoparticles: The impact of cationic lipid selection. J. Controlled Release 325:370-379.
Lu, H., Zhang, C., Albrecht, U., Shimizu, R., Wang, G., Bowman, K. D. 2013. Overexpression of a citrus NDR1 ortholog increases disease resistance in Arabidopsis. Front. Plant Sci. 4: Article 157:1-10.

Lück, S., Kreszies, T., Strickert, M., Schweizer, P., Kuhlmann, M., Douchkov, D. 2019. siRNA-Finder (si-Fi) Software for RNAi-Target Design and Off-Target Prediction. Front. Pl. Sci. 10: DOI=10.3389/fpls.2019.01023
Melnyk, CW., Molnar, A. and D. C. Baulcombe 2011. Intercellular and systemic movement of RNA silencing signals. The EMBO Journal 30:3553-3563.
Pardi, N., Hogan, M., Porter, F. et al. 2018. mRNA vaccines—a new era in vaccinology. Nat Rev Drug Discov 17, 261-279. https://doi.org/10.1038/nrd.2017.243.
Pérez SE, Gándola Y, Carlucci A M, González L, Turyn D, Bregni C. 2012. Formulation Strategies, Characterization, and In Vitro Evaluation of Lecithin-Based Nanoparticles for siRNA Delivery. J Drug Deliv. 2012; 2012:986265. doi: 10.1155/2012/986265.
Portt, L., Norman, G., Clapp, C., Greenwood, M., and Greenwood, M. T. Anti-apoptosis and cell survival: A review. Biochimica et Biophysica Acta 1813:238-259.
Reynolds, A., D. Leake, Q. Boese, S. Scaringe, W. S. Marshall, and A. Khvorova. 2004. Rational siRNA design for RNA interference. Nature Biotechnology 22:326-330.
Sarowar, S., Y. J. Kim, E. N. Kim, K. D. Kim, B. K. Hwang, R. Islam, and J. S. Shin. 2005. Overexpression of a pepper basic pathogenesis-related protein 1 gene in tobacco plants enhances resistance to heavy metal and pathogen stresses. Plant Cell Reports 24, no. 4:216-224.
Shi, Q., Febres, V. J., Jones, J. B., Moore, G. A. 2014. Responsiveness of different citrus genotypes to the Xanthomonas citri ssp. citri-derived pathogen-associated molecular pattern (PAMP) flg22 correlates with resistance to citrus canker. Molecular Plant Pathology. Article first published online; DOI: 10.1111/mpp.12206.
Shi, Q., Febres, V. J., Jones, J. B., Moore, G. A. 2013. Flg22 derived from Xanthomonas citri subsp. citri and ‘Candidatus Liberibacter asiaticus’ trigger similar defense responses in mandarin and grapefruit. Phytopathology. 103 (Suppl. 2): S2.132.
Stoutjesdijk P A1, Singh S P, Liu Q, Hurlstone C J, Waterhouse P A, Green A G. 2002. hpRNA-mediated targeting of the Arabidopsis FAD2 gene gives highly efficient and stable silencing. Plant Physiol. 129:1723-31.
Takayama, S., Sato, T., Krajewski, S., Kochel, K., Irie, S., Millan, J. A., and Reed, J. C. 1995. Cloning and functional analysis of BAG-1, a novel BCL-2 binding protein with anti-cell death activity. Cell 80, 279-284).
Van Loon L, Rep M, Pieterse C. 2006. Significance of inducible defense-related proteins in infected plants. Annu. Rev. Phytopathol. 44:135-162.
Varkonyi-Gasic, E., Wu, R., Wood, M. et al. 2007. Protocol: a highly sensitive RT-PCR method for detection and quantification of microRNAs. Plant Methods 3, 12. https://doi.org/10.1186/1746-4811-3-12
Watanabe N, Lam E (2008) BAX inhibitor-1 modulates endoplasmic reticulum stress mediated programmed cell death in Arabidopsis. J Biol Chem 283:3200-3210.
Williams, B., Kabbage, M., Britt, R., and Dickman, M. B. 2010. AtBAG7, an Arabidopsis Bcl-2-associated athano gene, resides in the endoplasmic reticulum and is involved in the unfolded protein response. Proc. Natl. Acad. Sci. USA 107:6088-6093.
Xu, P., Rogers, S. J., Roossinck, M. J., 2004. Expression of antiapoptotic genes bcl-xL and ced-9 in tomato enhances tolerance to viral-induced necrosis and abiotic stress. Proc. Natl. Acad. Sci. USA 101:15805-15810.
Zhang, S., Flores-Cruz, Z., Zhou, L., Kang, B. H., Fleites, L., Gooch, M. D., Wulff, N. A., Davis, M. J., Duan, Y., and Gabriel, D. W. 2011. Ca. Liberibacter asiaticus carries an excision plasmid prophage and a chromosomally integrated prophage that becomes lytic in plant infections. Molec. Plant-Microbe Interactions 24:458-468.

1. A recombinant nucleic acid molecule comprising a nucleic acid sequence comprising at least 15, 16, 17, 18, 19, 20, 21, 22, 23, 24 or 25 contiguous nucleotides of SEQ ID NO: 2.

2. A method of repressing, preventing or otherwise reducing a bacterial or fungal infection of a plant comprising expressing the nucleic acid molecule of claim 1 in said plant.

3. The method of claim 2, wherein said plant is a citrus tree.

4. The method of claim 2, wherein said infection is caused by a biotrophic plant pathogen.

5. The method of claim 2, wherein said infection is caused by a Liberibacter.

6. The method of claim 5 wherein said infection is caused by a Liberibacter infecting citrus.

7. The method of claim 5, wherein said infection is caused by Ca. Liberibacter asiaticus (Las).

8. A recombinant or completely synthetic nucleic acid molecule comprising a nucleic acid sequence comprising at least 15, 16, 17, 18, 19, 20, 21, 22, 23, 24 or 25 contiguous nucleotides of SEQ ID NO:2.

9. A recombinant or nonrecombinant nucleic acid molecule of claim 1 or 8, wherein said molecule is comprised of a dsRNA or siRNA.

10. A method of repressing, preventing or otherwise reducing a bacterial or fungal infection of a plant comprising topical application, injection or any form of introduction of the nucleic acid of claim 8 or 9 to said plant.

11. The method of claim 10, wherein the topical application comprises lecithin and/or gelatin formed into an emulsion.

12. The method of claim 10 or 11, wherein said plant is a citrus tree.

13. The method of claim 10 or 11, wherein said infection is caused by a biotrophic plant pathogen.

14. The method of claim 10 or 11, wherein said infection is caused by a Liberibacter.

15. The method of claim 14 wherein said infection is caused by a Liberibacter infecting citrus.

16. The method of claim 14, wherein said infection is caused by Ca. Liberibacter asiaticus (Las).

17. A self-amplifying mRNA (SAM) comprising a first nucleotide sequence encoding a RNA-dependent RNA polymerase (RdRp) operably linked to a second nucleotide sequence encoding a nucleotide sequence that forms a dsRNA in a plant cell, wherein the first nucleotide sequence is derived from a plant alphavirus.

18. The SAM of claim 17, wherein the second nucleotide sequence comprises at least 18 contiguous nucleotides of SEQ ID NO: 3

19. The SAM of claim 18, wherein the second nucleotide sequence is at least 25 bp.

20. The SAM of any of claims 17-19, wherein the second nucleotide sequence is SEQ ID NO: 3.

21. The SAM of any of claims 17-20, wherein the plant alphavirus is grapevine virus A.

22. The SAM of any of claims 17-21, wherein the first nucleotide sequence is SEQ ID NO: 4 or a nucleotide sequence comprising at least 80%, 85%, 90%, 95% or 98% sequence identity thereto.

23. A composition comprising the SAM of claim 17, 18, 19, 20, 21, or 22 formulated such that the SAM is encapsulated in a nanoemulsion.

24. The composition of claim 23, wherein the nanoemulsion comprises nanoparticles into which the SAM is contained.

25. The composition of claim 24, wherein the nanoemulsion comprises lecithin and/or gelatin.

26. A composition comprising the recombinant or nonrecombinant nucleic acid molecule of claim 8 formulated such that the recombinant or nonrecombinant nucleic acid molecule is encapsulated in a nanoemulstion.

27. The composition of claim 26, wherein the nanoemulsion comprises nanoparticle into which the recombinant or synthetic nucleic acid molecule is contained.

28. The composition of claim 26, wherein the nanoemulsion comprises lecithin and/or gelatin.

29. A method of repressing, preventing or otherwise reducing a bacterial or fungal infection of a plant comprising introducing into the plant the SAM of claim 17, 18, 19, 20, 21, or 22.

30. A method of repressing, preventing or otherwise reducing a bacterial or fungal infection of a plant comprising introducing into the plant the composition of claim 23.

31. A method of repressing, preventing or otherwise reducing a bacterial or fungal infection of a plant comprising introducing into the plant the SAM of claim of any of claims 26-28.


SEQUENCE LISTING (5 sequences)

SEQ ID NO: 1 - DNA, 5375 bp, Grapevine virus A
  source 1..5375: mol_type = genomic DNA
gaatatttaacttgattcccatcgattataagtgaacaaacgttgccagcacccacgctctgcaaagcacctgctaattcactaccatgtcgatatcagtatcctcccaacgtgtcgcagtctccaacctgtacacgaatggatctgaggagtcagttaaagctattaaggagttgaaaagcaaacggttattggaaaccgaaaccaggctagatggactatttgattactacattccagataccttaagagaaattcttacaggctatggtatggagttcagtgtccactctttccaaggacatgctcaccccgtaagtaagatgatagaaaaccacatgttgtatagagtagcaccaaattacttttctagtaatacattggtagttagttgtaaagagagcaagataaagcgcctacgtctgaagaatgcaaacaataggaatttgaacttcacacagtacaatagattggtgcatgcaaaccatcaccacagatacgaaaacgcattcagagaactcgacgttgggaatttgacaaatctcataaacaaggaagatcagagtgaatgcatattcatacatgatgaggttcaatactggagtctagacgagatgcaaaggttcttaggtagtctctccaaagtcgatagagtagtgtatagtatcatatacccatcagaggtcgaggctggatattcacaaagcctatttccagaggcgtacacttttgacctcaaagacaggagattagtatggtacccggacgggaaggctgaaggagcctatacgcagcctgtgaacccttggctgctcagatgctcaaaaacggaggattcgaaggggcgatcatggacgatcacaaagctccaaaccatcggagctcatcacctctttagtgcaataaagggcagctacttgacggaggaatcgtacaagtacgataacttcacgatcataaaccctaacgatgttctgaagggaaagagaggcgggaaaccactctatctacgggcccgcatgatcaagccaacacttctgtacctcttggctctgaagaaaagcgactccaactccgcagtcgcaaagctaaggatgctgagcagccgggaggagaacatggacgaggccctatttgtggcacaactcgcaaagcagataaaggacacggctctgtatgacaagatggggaaccctaacctgaggagtatactgtctgaatccttctatgatatcgcaggaagcctctttactcgcttattcaatcgccccgagtatgatgcgaggtgcctggagaaattcataaggagctgtgaaacgacagagatacatgtggaacggagatacatggaaggcataagaagaggggcctcattcaaggttcaaaatgtcatggactgggtcgaggacgacagcgccaacgcgctcagtgaggtgaacttcctcgacatcagctggaacgatagagtgtcagagccctatggcatcgaatgcattcacggagaagggagtaggataagggtcccactatccagaatactcagggcccacgaattgattgcaggggtccaaacagatgtggaaatcaacttcccgcggtatgtgtgctcctctagagccctcatacacttcaggcagtacctcatcaagcttgggaggttcagtttcatggagagcagggccatcaaggacatcgaagacatacaagcaggactcgaagagggggtgatcacagaggaagaagcggaactgcgcttattacccaccacaaagccgaagatcacagaaatccacatggatgatgacacgccaggaaccagtggcgagagtgatgtggagaagttcaaaagtgtgaggagcctgtgtcgtgaggaaatctactccgagaaactgaaagggcgcgaggtggcgttctatagcaggcacagcaaggagtacaaatacaatgggggctcccatcgttccctcggttgggatgaggctctaaatgagctcacacaggaactgggcctagatgatagctacgatcattgtctgatccaaagatacacggcagggggcagtataggattccacgcagatgatgaaccatgctacctaccagggggttccgtcgtgaccgtcaatttacacggcgacgctacttttgaggtgaaagagaatcaatccgggaagatcgagaagaaggagctacacgacggggatgtgtacgtgatgggtccaggaatgcaacaaactcacaagcacagagttacctctcacacggatggccgctgcagcatcacccttcggaacaaaacggtagattatgaagccagaaagggcgacgaggattctgagtatgaggaggacaaagctgaactggatgaaggcattgactacctacagaaaaatcaaggcaacatgtgctccctcaaagctttcgctgaccacatgcagttgagcaccccaagcgtcatagccatagtcaacggggcatcgccacaaaccctgagggaaatagaggatggcgggtacagtctggctacactagtcaacctctcaaaggcactcgattttccaatagctatacacggcgagagggggtacgctgagacaccagggtcttacaggcgtctgcacctgaagatcacatcagggcacgtggagcctttcgagggcgtgacaagcaaagggggctttagggaggccatgctgcttggtgacggcgtcggagtcgggcatttcagggtggacaaagccaaggccgaccgtctggctcaaagtttctacaatggaaacacgggggttctgctcggcaagtacaataaaggcaagatgcatacgggcgagatagaggagccaaaagaggttctaacggcctttggatttgcaggatcagggaaaagtcactggtgccaaaccatacttaaacactgttccgtggagaaagtgcttgtgattagcccaaggaaagtgctcagagatgattgggtagcaaaaatatccaagaagcatagagtggtaactttcgaggtggcattcatggacgactacggctgcaaagatatcgtgatagacgagatcggtcttctaccacctgggtacatagaccttgtgatagccgcacatcagccacgcaccctggtgctcctgggagatcctcttcaaagcacctaccacagtaaacgtgacaatgtggtcctggaggctagtcaagaggatgtcttcaatcgggtgagagggaaactaccatacttatgctactcacataggttacccagaaactgcaagctgttcgaaatagaatgcatgggggccgagagcgagaagagggtggtgtaccgctctaacaggctgaaagatgagcccaccatatgcgccaccagagcaatgaaggaagagaaggggtccgggtggtacaccgtcagtgagacgcaaggtctgagcttcaaatcatgcctcatttatctggacgagcactgggcaaagaaagaagatgaggacgtgatggtggccttaacccgctccagaggcgaaatcggcatacatgtaacccctgctctgaagaagaaactgatcaccaatgctaaaagcacactgctgaagaaagtactcaagggcgaaacctacagaagatcggagatagtggcaatggtccgaaagcacataccagagaccacagtgctgtttgaggaaagcagactggccgagacggtggactacgaggctaggctggctggcgacccctaccttaaatcccttctggccctgtacgacgagatagagatggaggacatagagatagaggagccggtgacactcgagcccaccaagacccatttagccctgagcacaaagatgaacgagcttgcaccatttgacctcaaagcaaaagagcaccgggagcaacacacagaggcagggcggaccgaacagatagacgagaatgggtaccagggagaagtgggtgaccccatgacccacaaggcgttgtacctgaggcatacatctgatgatacagctaccttcatgatgtcagtcaaaaagaggttgcgcttcagaaactatgaagccaacagaaggaagtacaagacttgccacggtatagggcaccaaatgttctcggtgttcaaggacacataccagctcaaagagattgactcactgcccgaactggaaaggtgcgaaatggagttcatgaagaagaggatagagaagagcaccgggcttatcgaaaagcatgccggaaggagtgacccagactggccgagcaattacctcaaaatattcctaaagcagcagacctgcacaaaaatggaaaagaggggggtggatgctaaggcgggacaaaccatcgcctgtttcgctcattcggtgctgtgtagattcgggcccattctgcgtcaaactgagaaagcgctacgggagctcctgcccgagaagctcatgatatactctcagaaaaagtacatggacttggataaatgggctaagacgtgggtagagagcatgatggggacggactccgactacgaggcattcgacagatcacaagacgagaaagtgctggacttggaggtggaggtcttgcgcttctttctatggcccgaagatttaatcagggagtacgaggagcttaagctgatgatgggatgtgcattaggcgacctggcggtgatgaggttctccggggaattcggcaccttcttcttcaacaccgtgtgcaacatggtgtttagttgtatgcgttaccacatagacaggaataccccgatgtgctttgcgggggatgatatgtattcgccaggcattctgagagtgaagaaggattacgaagccactctcgaccagttgacacttaaagccaaagtccatatatctgaggaacccctcttctgtgggtggaggatgagcccattcggaataatcaaggagccaaatctcatactcgacaggtggaagatagcgctgaggagcgggaatctatcactatgtttggtgaactatgcgatcgaggcaagctttgggtacaggttaagtgagcacctgtatgatgtgaatattgatgttgatgcacagcaagagctcgtgagggaaatagtgatcaaaaagcacttgctaccaaagaagatatctgatcttttcagcgaagacgagtgcgaacgtcacagcgacggggatgaagatttcttaagcaacgacgtcgcgagattgtacaggattgagtgaatttctaggacatggtagtgatagctgtgattcaggtagtgggtctctagggtctttgtcctatgtacagtgtgtcaatcttttgagtgatctaaagagtttagggtaccagagtatagatagcatactatatattttgggtggtggcgaggcggaacgattcga

SEQ ID NO: 2 - DNA, 493 bp, synthetic construct
  misc_feature 1..493: note = Chimeric sequence derived from SSADH and synthesized as dsRNA for field application
  source 1..493: mol_type = other DNA
cgtatttacaaacaaaaatcctgcaacttttgggatcaaagatcccaaatcgaatctttcatttcagttcagttgtcatcacgcgaaacagtaataaaagagtagtacaccaaacaaaggacgaaatagcaaatgtatcgtgtatgggaggaaaggaaacaaaagatgctatatcttcggcttttgatgcgtttaactctgcctgatcgccggatatttgttctatacagtggttctaaaaccctctgaatttctttgctcacgagcccacaggtgaggaagattacatttacatggctttggcgaaggtgtagtccagggtccactaattaataaagctgcaccacttctatgcttcaaaactgaggaggaagctatccatatggccaatgataccaatgcagggttagctgcttatatatttacaaacaatcttcaatggatgaattcctggaaataaaatatgtgtgcttgggagacatgaacagaaaat

SEQ ID NO: 3 - DNA, 1124 bp, synthetic construct
  misc_feature 1..1124: note = IPG-1M, synthetic based on SEQ ID NO:2 to encode both sense and antisense dsRNA with hairpin loop added
  source 1..1124: mol_type = other DNA
atgtcgatatcagtatcctcccaacgtgtcgcagtctccaacctgtacacgaatggatctgaggagtcagttaaagctattaaggagttgaaaagcaaacggttattggaaaccgaaaccatggggatcccgtatttacaaacaaaaatcctgcaacttttgggatcaaagatcccaaatcgaatctttcatttcagttcagttgtcatcacgcgaaacagtaataaaagagtagtacaccaaacaaaggacgaaatagcaaatgtatcgtgtatgggaggaaaggaaacaaaagatgctatatcttcggcttttgatgcgtttaactctgcctgatcgccggatatttgttctatacagtggttctaaaaccctctgaatttctttgctcacgagcccacaggtgaggaagattacatttacatggctttggcgaaggtgtagtccagggtccactaattaataaagctgcaccacttctatgcttcaaaactgaggaggaagctatccatatggccaatgataccaatcagggttagctgcttatatatttacaaacaatcttcaatggatgaattcctggaaataaaatatgtgtgcttgggagacatgaacagaaaataacaattgagaatttctgttcatgtctcccaagcacacatattttatttccaggaattcatccattgaagattgtttgtaaatatataagcagctaaccctgcattggtatcattggccatatggatagcttcctcctcagttttgaagcatagaagtggtgcagctttattaattagtggaccctggactacaccttcgccaaagccatgtaaatgtaatcttcctcacctgtgggctcgtgagcaaagaaattcagagggttttagaaccactgtatagaacaaatatccggcgatcaggcagagttaaacgcatcaaaagccgaagatatagcatcttttgtttcctttcctcccatacacgatacatttgctatttcgtcctttgtttggtgtactactcttttattactgtttcgcgtgatgacaactgaactgaaatgaaagattcgatttgggatctttgatcccaaaagttgcaggatttttgtttgtaaatac

SEQ ID NO: 4 - DNA, 9706 bp, synthetic construct
  misc_feature 1..9706: note = GVA-RNA-IPG1-MV4 plasmid designed to express an mRNA to produce a self-amplifying RNA that further expresses a dsRNA when introduced into plant cells.
  source 1..9706: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagattaatacgactcactatagtcgacgaatatttaacttgattcccatcgattataagtgaacaaacgttgccagcacccacgctctgcaaagcacctgctaattcactaccatgtcgatatcagtatcctcccaacgtgtcgcagtctccaacctgtacacgaatggatctgaggagtcagttaaagctattaaggagttgaaaagcaaacggttattggaaaccgaaaccaggctagatggactatttgattactacattccagataccttaagagaaattcttacaggctatggtatggagttcagtgtccactctttccaaggacatgctcaccccgtaagtaagatgatagaaaaccacatgttgtatagagtagcaccaaattacttttctagtaatacattggtagttagttgtaaagagagcaagataaagcgcctacgtctgaagaatgcaaacaataggaatttgaacttcacacagtacaatagattggtgcatgcaaaccatcaccacagatacgaaaacgcattcagagaactcgacgttgggaatttgacaaatctcataaacaaggaagatcagagtgaatgcatattcatacatgatgaggttcaatactggagtctagacgagatgcaaaggttcttaggtagtctctccaaagtcgatagagtagtgtatagtatcatatacccatcagaggtcgaggctggatattcacaaagcctatttccagaggcgtacacttttgacctcaaagacaggagattagtatggtacccggacgggaaggctgaaggagcctatacgcagcctgtgaacccttggctgctcagatgctcaaaaacggaggattcgaaggggcgatcatggacgatcacaaagctccaaaccatcggagctcatcacctctttagtgcaataaagggcagctacttgacggaggaatcgtacaagtacgataacttcacgatcataaaccctaacgatgttctgaagggaaagagaggcgggaaaccactctatctacgggcccgcatgatcaagccaacacttctgtacctcttggctctgaagaaaagcgactccaactccgcagtcgcaaagctaaggatgctgagcagccgggaggagaacatggacgaggccctatttgtggcacaactcgcaaagcagataaaggacacggctctgtatgacaagatggggaaccctaacctgaggagtatactgtctgaatccttctatgatatcgcaggaagcctctttactcgcttattcaatcgccccgagtatgatgcgaggtgcctggagaaattcataaggagctgtgaaacgacagagatacatgtggaacggagatacatggaaggcataagaagaggggcctcattcaaggttcaaaatgtcatggactgggtcgaggacgacagcgccaacgcgctcagtgaggtgaacttcctcgacatcagctggaacgatagagtgtcagagccctatggcatcgaatgcattcacggagaagggagtaggataagggtcccactatccagaatactcagggcccacgaattgattgcaggggtccaaacagatgtggaaatcaacttcccgcggtatgtgtgctcctctagagccctcatacacttcaggcagtacctcatcaagcttgggaggttcagtttcatggagagcagggccatcaaggacatcgaagacatacaagcaggactcgaagagggggtgatcacagaggaagaagcggaactgcgcttattacccaccacaaagccgaagatcacagaaatccacatggatgatgacacgccaggaaccagtggcgagagtgatgtggagaagttcaaaagtgtgaggagcctgtgtcgtgaggaaatctactccgagaaactgaaagggcgcgaggtggcgttctatagcaggcacagcaaggagtacaaatacaatgggggctcccatcgttccctcggttgggatgaggctctaaatgagctcacacaggaactgggcctagatgatagctacgatcattgtctgatccaaagatacacggcagggggcagtataggattccacgcagatgatgaaccatgctacctaccagggggttccgtcgtgaccgtcaatttacacggcgacgctacttttgaggtgaaagagaatcaatccgggaagatcgagaagaaggagctacacgacggggatgtgtacgtgatgggtccaggaatgcaacaaactcacaagcacagagttacctctcacacggatggccgctgcagcatcacccttcggaacaaaacggtagattatgaagccagaaagggcgacgaggattctgagtatgaggaggacaaagctgaactggatgaaggcattgactacctacagaaaaatcaaggcaacatgtgctccctcaaagctttcgctgaccacatgcagttgagcaccccaagcgtcatagccatagtcaacggggcatcgccacaaaccctgagggaaatagaggatggcgggtacagtctggctacactagtcaacctctcaaaggcactcgattttccaatagctatacacggcgagagggggtacgctgagacaccagggtcttacaggcgtctgcacctgaagatcacatcagggcacgtggagcctttcgagggcgtgacaagcaaagggggctttagggaggccatgctgcttggtgacggcgtcggagtcgggcatttcagggtggacaaagccaaggccgaccgtctggctcaaagtttctacaatggaaacacgggggttctgctcggcaagtacaataaaggcaagatgcatacgggcgagatagaggagccaaaagaggttctaacggcctttggatttgcaggatcagggaaaagtcactggtgccaaaccatacttaaacactgttccgtggagaaagtgcttgtgattagcccaaggaaagtgctcagagatgattgggtagcaaaaatatccaagaagcatagagtggtaactttcgaggtggcattcatggacgactacggctgcaaagatatcgtgatagacgagatcggtcttctaccacctgggtacatagaccttgtgatagccgcacatcagccacgcaccctggtgctcctgggagatcctcttcaaagcacctaccacagtaaacgtgacaatgtggtcctggaggctagtcaagaggatgtcttcaatcgggtgagagggaaactaccatacttatgctactcacataggttacccagaaactgcaagctgttcgaaatagaatgcatgggggccgagagcgagaagagggtggtgtaccgctctaacaggctgaaagatgagcccaccatatgcgccaccagagcaatgaaggaagagaaggggtccgggtggtacaccgtcagtgagacgcaaggtctgagcttcaaatcatgcctcatttatctggacgagcactgggcaaagaaagaagatgaggacgtgatggtggccttaacccgctccagaggcgaaatcggcatacatgtaacccctgctctgaagaagaaactgatcaccaatgctaaaagcacactgctgaagaaagtactcaagggcgaaacctacagaagatcggagatagtggcaatggtccgaaagcacataccagagaccacagtgctgtttgaggaaagcagactggccgagacggtggactacgaggctaggctggctggcgacccctaccttaaatcccttctggccctgtacgacgagatagagatggaggacatagagatagaggagccggtgacactcgagcccaccaagacccatttagccctgagcacaaagatgaacgagcttgcaccatttgacctcaaagcaaaagagcaccgggagcaacacacagaggcagggcggaccgaacagatagacgagaatgggtaccagggagaagtgggtgaccccatgacccacaaggcgttgtacctgaggcatacatctgatgatacagctaccttcatgatgtcagtcaaaaagaggttgcgcttcagaaactatgaagccaacagaaggaagtacaagacttgccacggtatagggcaccaaatgttctcggtgttcaaggacacataccagctcaaagagattgactcactgcccgaactggaaaggtgcgaaatggagttcatgaagaagaggatagagaagagcaccgggcttatcgaaaagcatgccggaaggagtgacccagactggccgagcaattacctcaaaatattcctaaagcagcagacctgcacaaaaatggaaaagaggggggtggatgctaaggcgggacaaaccatcgcctgtttcgctcattcggtgctgtgtagattcgggcccattctgcgtcaaactgagaaagcgctacgggagctcctgcccgagaagctcatgatatactctcagaaaaagtacatggacttggataaatgggctaagacgtgggtagagagcatgatggggacggactccgactacgaggcattcgacagatcacaagacgagaaagtgctggacttggaggtggaggtcttgcgcttctttctatggcccgaagatttaatcagggagtacgaggagcttaagctgatgatgggatgtgcattaggcgacctggcggtgatgaggttctccggggaattcggcaccttcttcttcaacaccgtgtgcaacatggtgtttagttgtatgcgttaccacatagacaggaataccccgatgtgctttgcgggggatgatatgtattcgccaggcattctgagagtgaagaaggattacgaagccactctcgaccagttgacacttaaagccaaagtccatatatctgaggaacccctcttctgtgggtggaggatgagcccattcggaataatcaaggagccaaatctcatactcgacaggtggaagatagcgctgaggagcgggaatctatcactatgtttggtgaactatgcgatcgaggcaagctttgggtacaggttaagtgagcacctgtatgatgtgaatattgatgttgatgcacagcaagagctcgtgagggaaatagtgatcaaaaagcacttgctaccaaagaagatatctgatcttttcagcgaagacgagtgcgaacgtcacagcgacggggatgaagatttcttaagcaacgacgtcgcgagattgtacaggattgagtgaatttctaggacatggtagtgatagctgtgattcaggtagtgggtctctagggtctttgtcctatgtacagtgtgtcaatcttttgagtgatctaaagagtttagggtaccagagtatagatagcatactatatattttgggtggtggcgaggcggaacgattcgagatctatcgaatcttccggaggcacggaatcgggatcggagaggctttacagctaggcgtcaaaaagagtttgtgcaacagtccaagaagtctactggcaatactagacgacttgttgtctagacttgggagaggcagcgctttccttccaagtgatctgggggcagtcaaaggtgagctggtagcaacgttccactcgtccagattaagtgtagacctttacgttaataacaagaaggttgtcacgcgatcagtgcaagcggaagacagaatatttaacttgattcccatcgattataagtgaacaaacgttgccagcacccacgctctgcaaagcacctgctaattcactaccatgtcgatatcagtatcctcccaacgtgtcgcagtctccaacctgtacacgaatggatctgaggagtcagttaaagctattaaggagttgaaaagcaaacggttattggaaaccgaaaccatggggatcccgtatttacaaacaaaaatcctgcaacttttgggatcaaagatcccaaatcgaatctttcatttcagttcagttgtcatcacgcgaaacagtaataaaagagtagtacaccaaacaaaggacgaaatagcaaatgtatcgtgtatgggaggaaaggaaacaaaagatgctatatcttcggcttttgatgcgtttaactctgcctgatcgccggatatttgttctatacagtggttctaaaaccctctgaatttctttgctcacgagcccacaggtgaggaagattacatttacatggctttggcgaaggtgtagtccagggtccactaattaataaagctgcaccacttctatgcttcaaaactgaggaggaagctatccatatggccaatgataccaatcagggttagctgcttatatatttacaaacaatcttcaatggatgaattcctggaaataaaatatgtgtgcttgggagacatgaacagaaaataacaattgagaatttctgttcatgtctcccaagcacacatattttatttccaggaattcatccattgaagattgtttgtaaatatataagcagctaaccctgcattggtatcattggccatatggatagcttcctcctcagttttgaagcatagaagtggtgcagctttattaattagtggaccctggactacaccttcgccaaagccatgtaaatgtaatcttcctcacctgtgggctcgtgagcaaagaaattcagagggttttagaaccactgtatagaacaaatatccggcgatcaggcagagttaaacgcatcaaaagccgaagatatagcatcttttgtttcctttcctcccatacacgatacatttgctatttcgtcctttgtttggtgtactactcttttattactgtttcgcgtgatgacaactgaactgaaatgaaagattcgatttgggatctttgatcccaaaagttgcaggatttttgtttgtaaatacgcggccgcccgggagggcgaattctgcagatgggtcgtctatcctagctaaaaagtaaagtataacttaataataaagcgagctaggttgtcacacgaagacaaaaaaaaaaaaaaaaacgcgtatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaagagtttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcaagcaagacgtttcccgttgaatatggctcattttagcttccttagctcctgaaaatctcgataactcaaaaaatacgcccggtagtgatcttatttcattatggtgaaagttggaacctcttacgtgccgatcacacatttccccgaaaagtgccacctgacgtctaagaaaccattattatcatgacattaacctataaaaataggcgtatcacgaggccctttcgtc

SEQ ID NO: 5 - DNA, 9400 bp, synthetic construct
  misc_feature 1..9400: note = GVA-RNA-CSE-eGFPv2 plasmid designed to express an mRNA to produce a self-amplifying RNA that further expresses enhanced GFP protein when introduced into plant cells.
  source 1..9400: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagattaatacgactcactatagtcgacgaatatttaacttgattcccatcgattataagtgaacaaacgttgccagcacccacgctctgcaaagcacctgctaattcactaccatgtcgatatcagtatcctcccaacgtgtcgcagtctccaacctgtacacgaatggatctgaggagtcagttaaagctattaaggagttgaaaagcaaacggttattggaaaccgaaaccaggctagatggactatttgattactacattccagataccttaagagaaattcttacaggctatggtatggagttcagtgtccactctttccaaggacatgctcaccccgtaagtaagatgatagaaaaccacatgttgtatagagtagcaccaaattacttttctagtaatacattggtagttagttgtaaagagagcaagataaagcgcctacgtctgaagaatgcaaacaataggaatttgaacttcacacagtacaatagattggtgcatgcaaaccatcaccacagatacgaaaacgcattcagagaactcgacgttgggaatttgacaaatctcataaacaaggaagatcagagtgaatgcatattcatacatgatgaggttcaatactggagtctagacgagatgcaaaggttcttaggtagtctctccaaagtcgatagagtagtgtatagtatcatatacccatcagaggtcgaggctggatattcacaaagcctatttccagaggcgtacacttttgacctcaaagacaggagattagtatggtacccggacgggaaggctgaaggagcctatacgcagcctgtgaacccttggctgctcagatgctcaaaaacggaggattcgaaggggcgatcatggacgatcacaaagctccaaaccatcggagctcatcacctctttagtgcaataaagggcagctacttgacggaggaatcgtacaagtacgataacttcacgatcataaaccctaacgatgttctgaagggaaagagaggcgggaaaccactctatctacgggcccgcatgatcaagccaacacttctgtacctcttggctctgaagaaaagcgactccaactccgcagtcgcaaagctaaggatgctgagcagccgggaggagaacatggacgaggccctatttgtggcacaactcgcaaagcagataaaggacacggctctgtatgacaagatggggaaccctaacctgaggagtatactgtctgaatccttctatgatatcgcaggaagcctctttactcgcttattcaatcgccccgagtatgatgcgaggtgcctggagaaattcataaggagctgtgaaacgacagagatacatgtggaacggagatacatggaaggcataagaagaggggcctcattcaaggttcaaaatgtcatggactgggtcgaggacgacagcgccaacgcgctcagtgaggtgaacttcctcgacatcagctggaacgatagagtgtcagagccctatggcatcgaatgcattcacggagaagggagtaggataagggtcccactatccagaatactcagggcccacgaattgattgcaggggtccaaacagatgtggaaatcaacttcccgcggtatgtgtgctcctctagagccctcatacacttcaggcagtacctcatcaagcttgggaggttcagtttcatggagagcagggccatcaaggacatcgaagacatacaagcaggactcgaagagggggtgatcacagaggaagaagcggaactgcgcttattacccaccacaaagccgaagatcacagaaatccacatggatgatgacacgccaggaaccagtggcgagagtgatgtggagaagttcaaaagtgtgaggagcctgtgtcgtgaggaaatctactccgagaaactgaaagggcgcgaggtggcgttctatagcaggcacagcaaggagtacaaatacaatgggggctcccatcgttccctcggttgggatgaggctctaaatgagctcacacaggaactgggcctagatgatagctacgatcattgtctgatccaaagatacacggcagggggcagtataggattccacgcagatgatgaaccatgctacctaccagggggttccgtcgtgaccgtcaatttacacggcgacgctacttttgaggtgaaagagaatcaatccgggaagatcgagaagaaggagctacacgacggggatgtgtacgtgatgggtccaggaatgcaacaaactcacaagcacagagttacctctcacacggatggccgctgcagcatcacccttcggaacaaaacggtagattatgaagccagaaagggcgacgaggattctgagtatgaggaggacaaagctgaactggatgaaggcattgactacctacagaaaaatcaaggcaacatgtgctccctcaaagctttcgctgaccacatgcagttgagcaccccaagcgtcatagccatagtcaacggggcatcgccacaaaccctgagggaaatagaggatggcgggtacagtctggctacactagtcaacctctcaaaggcactcgattttccaatagctatacacggcgagagggggtacgctgagacaccagggtcttacaggcgtctgcacctgaagatcacatcagggcacgtggagcctttcgagggcgtgacaagcaaagggggctttagggaggccatgctgcttggtgacggcgtcggagtcgggcatttcagggtggacaaagccaaggccgaccgtctggctcaaagtttctacaatggaaacacgggggttctgctcggcaagtacaataaaggcaagatgcatacgggcgagatagaggagccaaaagaggttctaacggcctttggatttgcaggatcagggaaaagtcactggtgccaaaccatacttaaacactgttccgtggagaaagtgcttgtgattagcccaaggaaagtgctcagagatgattgggtagcaaaaatatccaagaagcatagagtggtaactttcgaggtggcattcatggacgactacggctgcaaagatatcgtgatagacgagatcggtcttctaccacctgggtacatagaccttgtgatagccgcacatcagccacgcaccctggtgctcctgggagatcctcttcaaagcacctaccacagtaaacgtgacaatgtggtcctggaggctagtcaagaggatgtcttcaatcgggtgagagggaaactaccatacttatgctactcacataggttacccagaaactgcaagctgttcgaaatagaatgcatgggggccgagagcgagaagagggtggtgtaccgctctaacaggctgaaagatgagcccaccatatgcgccaccagagcaatgaaggaagagaaggggtccgggtggtacaccgtcagtgagacgcaaggtctgagcttcaaatcatgcctcatttatctggacgagcactgggcaaagaaagaagatgaggacgtgatggtggccttaacccgctccagaggcgaaatcggcatacatgtaacccctgctctgaagaagaaactgatcaccaatgctaaaagcacactgctgaagaaagtactcaagggcgaaacctacagaagatcggagatagtggcaatggtccgaaagcacataccagagaccacagtgctgtttgaggaaagcagactggccgagacggtggactacgaggctaggctggctggcgacccctaccttaaatcccttctggccctgtacgacgagatagagatggaggacatagagatagaggagccggtgacactcgagcccaccaagacccatttagccctgagcacaaagatgaacgagcttgcaccatttgacctcaaagcaaaagagcaccgggagcaacacacagaggcagggcggaccgaacagatagacgagaatgggtaccagggagaagtgggtgaccccatgacccacaaggcgttgtacctgaggcatacatctgatgatacagctaccttcatgatgtcagtcaaaaagaggttgcgcttcagaaactatgaagccaacagaaggaagtacaagacttgccacggtatagggcaccaaatgttctcggtgttcaaggacacataccagctcaaagagattgactcactgcccgaactggaaaggtgcgaaatggagttcatgaagaagaggatagagaagagcaccgggcttatcgaaaagcatgccggaaggagtgacccagactggccgagcaattacctcaaaatattcctaaagcagcagacctgcacaaaaatggaaaagaggggggtggatgctaaggcgggacaaaccatcgcctgtttcgctcattcggtgctgtgtagattcgggcccattctgcgtcaaactgagaaagcgctacgggagctcctgcccgagaagctcatgatatactctcagaaaaagtacatggacttggataaatgggctaagacgtgggtagagagcatgatggggacggactccgactacgaggcattcgacagatcacaagacgagaaagtgctggacttggaggtggaggtcttgcgcttctttctatggcccgaagatttaatcagggagtacgaggagcttaagctgatgatgggatgtgcattaggcgacctggcggtgatgaggttctccggggaattcggcaccttcttcttcaacaccgtgtgcaacatggtgtttagttgtatgcgttaccacatagacaggaataccccgatgtgctttgcgggggatgatatgtattcgccaggcattctgagagtgaagaaggattacgaagccactctcgaccagttgacacttaaagccaaagtccatatatctgaggaacccctcttctgtgggtggaggatgagcccattcggaataatcaaggagccaaatctcatactcgacaggtggaagatagcgctgaggagcgggaatctatcactatgtttggtgaactatgcgatcgaggcaagctttgggtacaggttaagtgagcacctgtatgatgtgaatattgatgttgatgcacagcaagagctcgtgagggaaatagtgatcaaaaagcacttgctaccaaagaagatatctgatcttttcagcgaagacgagtgcgaacgtcacagcgacggggatgaagatttcttaagcaacgacgtcgcgagattgtacaggattgagtgaatttctaggacatggtagtgatagctgtgattcaggtagtgggtctctagggtctttgtcctatgtacagtgtgtcaatcttttgagtgatctaaagagtttagggtaccagagtatagatagcatactatatattttgggtggtggcgaggcggaacgattcgagatctatcgaatcttccggaggcacggaatcgggatcggagaggctttacagctaggcgtcaaaaagagtttgtgcaacagtccaagaagtctactggcaatactagacgacttgttgtctagacttgggagaggcagcgctttccttccaagtgatctgggggcagtcaaaggtgagctggtagcaacgttccactcgtccagattaagtgtagacctttacgttaataacaagaaggttgtcacgcgatcagtgcaagcggaagacagaatatttaacttgattcccatcgattataagtgaacaaacgttgccagcacccacgctctgcaaagcacctgctaattcactaccatgtcgatatcagtatcctcccaacgtgtcgcagtctccaacctgtacacgaatggatctgaggagtcagttaaagctattaaggagttgaaaagcaaacggttattggaaaccgaaaccatggggatcctgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaacccgggtcgtctatcctagctaaaaagtaaagtataacttaataataaagcgagctaggttgtcacacgaagacaaaaaaaaaaaaaaaaacgcgtatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaagagtttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcaagcaagacgtttcccgttgaatatggctcattttagcttccttagctcctgaaaatctcgataactcaaaaaatacgcccggtagtgatcttatttcattatggtgaaagttggaacctcttacgtgccgatcacacatttccccgaaaagtgccacctgacgtctaagaaaccattattatcatgacattaacctataaaaataggcgtatcacgaggccctttcgtc